SEQUENCE LISTING (16 sequences)

SEQ ID NO: 1 - DNA, 2733 bp, Homo sapiens
  source 1..2733: mol_type = other DNA
ctcgcgaggccggctaggcccgaatgtcgttagccgtggggaaagatggcggaaaatttaaaaggctgcagcgtgtgttgcaagtcttcttggaatcagctgcaggacctgtgccgcctggccaagctctcctgccctgccctcggtatctctaagaggaacctctatgactttgaagtcgagtacctgtgcgattacaagaagatccgcgaacaggaatattacctggtgaaatggcgtggatatccagactcagagagcacctgggagccacggcagaatctcaagtgtgtgcgtatcctcaagcagttccacaaggacttagaaagggagctgctccggcggcaccaccggtcaaagaccccccggcacctggacccaagcttggccaactacctggtgcagaaggccaagcagaggcgggcgctccgtcgctgggagcaggagctcaatgccaagcgcagccatctgggacgcatcactgtagagaatgaggtggacctggacggccctccgcgggccttcgtgtacatcaatgagtaccgtgttggtgagggcatcaccctcaaccaggtggctgtgggctgcgagtgccaggactgtctgtgggcacccactggaggctgctgcccgggggcgtcactgcacaagtttgcctacaatgaccagggccaggtgcggcttcgagccgggctgcccatctacgagtgcaactcccgctgccgctgcggctatgactgcccaaatcgtgtggtacagaagggtatccgatatgacctctgcatcttccggacggatgatgggcgtggctggggcgtccgcaccctggagaagattcgcaagaacagcttcgtcatggagtacgtgggagagatcattacctcagaggaggcagagcggcggggccagatctacgaccgtcagggcgccacctacctctttgacctggactacgtggaggacgtgtacaccgtggatgccgcctactatggcaacatctcccactttgtcaaccacagttgtgaccccaacctgcaggtgtacaacgtcttcatagacaaccttgacgagcggctgccccgcatcgctttctttgccacaagaaccatccgggcaggcgaggagctcacctttgattacaacatgcaagtggaccccgtggacatggagagcacccgcatggactccaactttggcctggctgggctccctggctcccctaagaagcgggtccgtattgaatgcaagtgtgggactgagtcctgccgcaaatacctcttctagcccttagaagtctgaggccagactgactgagggggcctgaagctacatgcacctcccccactgctgccctcctgtcgagaatgactgccagggcctcgcctgcctccacctgcccccacctgctcctacctgctctacgttcagggctgtggccgtggtgaggaccgactccaggagtcccctttccctgtcccagccccatctgtgggttgcacttacaaacccccacccaccttcagaaatagtttttcaacatcaagactctctgtcgttgggattcatggcctattaaggaggtccaaggggtgagtcccaacccagccccagaatatatttgtttttgcacctgcttctgcctggagattgaggggtctgctgcaggcctcctccctgctgccccaaaggtatggggaagcaaccccagagcaggcagacatcagaggccagagtgcctagcccgacatgaagctggttccccaaccacagaaactttgtactagtgaaagaaaggggtccctggcctacgggctgaggctggtttctgctcgtgcttacagtgctgggtagtgttggccctaagagctgtagggtctcttcttcagggctgcatatctgagaagtggatgcccacatgccactggaagggaagtgggtgtccatgggccactgagcagtgagaggaaggcagtgcagagctggccagccctggaggtaggctgggaccaagctctgccttcacagtgcagtgaaggtacctagggctcttgggagctctgcggttgctaggggccctgacctggggtgtcatgaccgctgacaccactcagagctggaaccaagatctagatagtccgtagatagcacttaggacaagaatgtgcattgatggggtggtgatgaggtgccaggcactaggtagagcacctggtccacgtggattgtctcagggaagccttgaaaaccacggaggtggatgccaggaaagggcccatgtggcagaaggcaaagtacaggccaagaattgggggtgggggagatggcttccccactatgggatgacgaggcgagagggaagcccttgctgcctgccattcccagaccccagccctttgtgctcaccctggttccactggtctcaaaagtcacctgcctacaaatgtacaaaaggcgaaggttctgatggctgccttgctccttgctcccccaccccctgtgaggacttctctaggaagtccttcctgactacctgtgcccagagtgcccctacatgagactgtatgccctgctatcagatgccagatctatgtgtctgtctgtgtgtccatcccgccggccccccagactaacctccaggcatggactgaatctggttctcctcttgtacacccctcaaccctatgcagcctggagtgggcatcaataaaatgaactgtcgactgaaaaaaaaaa

SEQ ID NO: 2 - protein, 412 aa, Homo sapiens
MAENLKGCSVCCKSSWNQLQDLCRLAKLSCPALGISKRNLYDFEVEYLCDYKKIREQEYYLVKWRGYPDSESTWEPRQNLKCVRILKQFHKDLERELLRRHHRSKTPRHLDPSLANYLVQKAKQRRALRRWEQELNAKRSHLGRITVENEVDLDGPPRAFVYINEYRVGEGITLNQVAVGCECQDCLWAPTGGCCPGASLHKFAYNDQGQVRLRAGLPIYECNSRCRCGYDCPNRVVQKGIRYDLCIFRTDDGRGWGVRTLEKIRKNSFVMEYVGEIITSEEAERRGQIYDRQGATYLFDLDYVEDVYTVDAAYYGNISHFVNHSCDPNLQVYNVFIDNLDERLPRIAFFATRTIRAGEELTFDYNMQVDPVDMESTRMDSNFGLAGLPGSPKKRVRIECKCGTESCRKYLF

SEQ ID NO: 3 - protein, 90 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSP

SEQ ID NO: 4 - protein, 164 aa, Homo sapiens
  SITE 61..89: note = ITAM1
  SITE 100..128: note = ITAM2
  SITE 131..159: note = ITAM3
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 645 aa, Homo sapiens
  DOMAIN 1..42: note = Extracellular domain
MPIWKFPDEEGACQPCPINCTHSCVDLDDKGCPAEQRASPLTSIVSAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERAKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 6 - protein, 42 aa, Homo sapiens
MPIWKFPDEEGACQPCPINCTHSCVDLDDKGCPAEQRASPLT

SEQ ID NO: 7 - protein, 5 aa, synthetic construct
  REGION 1..5: note = p95HER2 HCDR1
DFGMS

SEQ ID NO: 8 - protein, 17 aa, synthetic construct
  REGION 1..17: note = p95HER2 HCDR2
TINTNGGTTHYPDNVKG

SEQ ID NO: 9 - protein, 5 aa, synthetic construct
  REGION 1..5: note = p95HER2 HCDR3
EGLDY

SEQ ID NO: 10 - protein, 11 aa, synthetic construct
  REGION 1..11: note = p95HER2 LCDR1
KASQSVGTAVA

SEQ ID NO: 11 - protein, 7 aa, synthetic construct
  REGION 1..7: note = p95HER2 LCDR2
SASNRFT

SEQ ID NO: 12 - protein, 9 aa, synthetic construct
  REGION 1..9: note = p95HER2 LCDR3
QQYSTYPLA

SEQ ID NO: 13 - protein, 114 aa, synthetic construct
  REGION 1..114: note = p95HER2 VH
EVQLVESGGGIVQPGGSLKLSCAASGFTFNDFGMSWIRQTPDKRLELVATINTNGGTTHYPDNVKGRFSISRDNAKKFVYLQMSSLKSDDTAIYYCPREGLDYWGQGTTLTVSS

SEQ ID NO: 14 - protein, 107 aa, synthetic construct
  REGION 1..107: note = p95HER2 VL
DIVLTQSQKFMSTSVGDRVSIICKASQSVGTAVAWYQLKAGQSPKLLIYSASNRFTGVPDRFTGSGSGTDFTLTISNVQSEDLADYFCQQYSTYPLAFGAGTKLELK

SEQ ID NO: 15 - protein, 7 aa, Homo sapiens
PIWKFPD

SEQ ID NO: 16 - RNA, 105 nt, synthetic construct
  misc_feature 1..105: note = Synethic polynucleotide
  misc_feature 2: note = n is 2'-O-methyl 3'phosphorothioate
  misc_feature 4: note = n is 2'-O-methyl 3'phosphorothioate
  misc_feature 6: note = n is 2'-O-methyl 3'phosphorothioate
  misc_feature 100: note = n is 2'-O-methyl 3'phosphorothioate
  misc_feature 102: note = n is 2'-O-methyl 3'phosphorothioate
  misc_feature 104: note = n is 2'-O-methyl 3'phosphorothioate
  source 1..105: mol_type = other RNA
cnangnggttctggatatctgtgttttagagctagaaatagcaagttaaaataaggctagtccgttatcaacttgaaaaagtggcaccgagtcggtgctntntnt

(19) United States
(12) Patent Application Publication
(10) Pub. No.: US 2025/0388906 A1
(43) Pub. Date: Dec. 25, 2025

(54) IMMUNE CELLS DEFECTIVE FOR SUV39H1

(71) Applicants: MNEMO THERAPEUTICS, Paris (FR); INSTITUT CURIE, Paris (FR); INSERM, Paris (FR)

(72) Inventors: Sebastian AMIGORENA, Paris (FR); Michael SAITAKIS, Paris (FR); Sheila LOPEZ-COBO, Paris (FR); Jaime Rodrigo FUENTEALBA, Paris (FR)

(21) Appl. No.: 19/227,105

(22) Filed: Jun. 3, 2025

Related U.S. Application Data
(63) Continuation of application No. 17/629,256, filed on Jan. 21, 2022, now abandoned.
(60) Provisional application No. 62/877,789, filed on Jul. 23, 2019.
(60) Provisional application No. 62/978,936, filed on Feb. 20, 2020.
(60) Provisional application No. 63/048,328, filed on Jul. 6, 2020.

(51) Int. Cl. C12N 15/113; A61K 40/11; A61K 40/31; A61K 40/42; C07K 14/725; C12N 5/0783

(52) U.S. Cl. CPC C12N 15/1138; A61K 40/11; A61K 40/31; A61K 40/4202; A61K 40/4211; C07K 14/7051; C12N 5/0636; A61K 2239/38; A61K 2239/48; C07K 2319/03; C12N 2310/20; C12N 2310/315; C12N 2310/321

(57) ABSTRACT

The present invention relates to an improved immune cell expressing an antigen-specific receptor such as a CAR or TCR, in which SUV39H1 is inactivated, optionally combined with disruption of the TRAC locus and/or deletion of one or more ITAMs. The invention also provides compositions comprising such cells, methods of producing such cells, and uses of such cells in adoptive cell therapy, e.g. in cancer or inflammatory diseases.

### RELATED APPLICATIONS

This application is a continuation of U.S. patent application Ser. No. 17/629,256, filed Jan. 21, 2022, which is the U.S. National Phase of International Patent Application No. PCT/EP2020/070845, filed Jul. 23, 2020, which claims priority to U.S. Provisional Patent Application Nos. 63/048,328, filed Jul. 6, 2020, 62/978,936, filed Feb. 20, 2020 and 62/877,789, filed Jul. 23, 2019, which are hereby incorporated by reference in their entirety.

### INCORPORATION BY REFERENCE OF SEQUENCE LISTING

This application includes, as a separate part of disclosure, a Sequence Listing in computer-readable form (filename: 1919CON.xml; 26,385 bytes; created Jun. 3, 2025). The contents of the Sequence Listing XML file are incorporated herein by reference.

### FIELD OF THE INVENTION

The present invention relates to the field of adoptive cell therapy. The present invention provides immune cells defective for SUV39H1 with enhanced properties.

### INTRODUCTION

Adoptive T cell therapy (ATCT) using T cells armed with recombinant T Cell Receptor (TCR) and Chimeric Antigen Receptor (CAR) technologies is emerging as a powerful cancer therapy alternative (Lim WA & June CH. 2018. Cell 168(4):724-740). Efficient engraftment, long-term persistence and reduced exhaustion of the therapeutic T cells correlates with positive therapeutic outcomes. Additionally, the increased persistence of adoptively transferred cells appears to be dependent upon the acquisition of central memory T cell (TCM) populations (Powell DJ et al., Blood. 2005; 105(1):241-50; Huang J, Khong HT et al. J Immunother. 2005; 28:258-267).

Upon activation, T cells progress in an irreversible linear fashion towards an effector (TE) phenotype (Mahnke YD et al., Eur J Immunol. 2013; 43:2797-2809; Farber DL. Semin Immunol. 2009; 21:84-91). Mitogenic activation for retroviral or lentiviral transduction, therefore, drives differentiation of T cells from a naïve towards a TE phenotype. In combination with ex-vivo culture protocols to expand transduced T cell numbers to those required for clinical application (about 109-1011), T cells are driven towards a more differentiated phenotype, which is sub-optimal for systemic persistence. A major obstacle for the successful cell-based therapy of solid tumors is the exhaustion of activated T cells, which decreases their ability to proliferate and destroy target cells. PD-1 blockade can restore T cell function at an early stage but the rescue may be incomplete or transient (Sen DR, et al. 2016. Science 354(6316):1165-1169; Pauken KE, et al. 2016. Science 354(6316):1160-1165). Moreover, the immunosuppressive microenvironment in the tumor mediates T cell exhaustion (Joyce JA, Fearon DT. 2015. Science 348(6230):74-80).

There remains a need in the art for modified or engineered T cells with improved properties for adoptive cell therapy.

### SUMMARY OF THE INVENTION

Immune cells, particularly T-cells, in which SUV39H1 has been inactivated or inhibited exhibit an enhanced central memory phenotype, enhanced survival and persistence after adoptive transfer, and reduced exhaustion. In particular, such cells accumulate and re-program with increased efficiency into longed-lived central memory T cells. Such cells are more efficient at inducing tumor cell rejection and display enhanced efficacy for treating cancer.

In one aspect, the disclosure provides a modified immune cell wherein the SUV39H1 gene is inactivated or inhibited, said cell comprising a T cell receptor (TCR) alpha constant region gene inactivated by the insertion of a nucleic acid sequence encoding an antigen-specific receptor that specifically binds to an antigen. The insertion of the nucleic acid sequence may reduce endogenous TCR expression by at least about 75%, 80%, 85%, 90% or 95%. For example, the nucleic acid encoding the antigen-specific receptor may be heterologous to the immune cell and operatively linked to an endogenous promoter of the T-cell receptor such that its expression is under control of the endogenous promoter. The antigen-specific receptor may be a chimeric antigen receptor (CAR) or a heterologous TCR. In some embodiments, the nucleic acid encoding a CAR is operatively linked to an endogenous TRAC promoter. Examples of antigens to which the antigen-specific receptor binds, preferably with a binding affinity KD of 10-7 M or 10-8 M or less, include orphan tyrosine kinase receptor ROR1, tEGFR, Her2, p95HER2, LI-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, BCMA, Lewis Y, MAGE-A1, mesothelin, MUC1, MUC16, PSCA, NKG2D Ligands, NY-ESO-1, MART-1, gplOO, oncofetal antigen, TAG72, VEGF-R2, carcinoembryonic antigen (CEA), prostate specific antigen (PSMA), estrogen receptor, progesterone receptor, ephrinB2, CD 123, CS-1, c-Met, GD-2, MAGE A3, CE7, or Wilms Tumor 1 (WT-1).

In another aspect, the disclosure provides a modified immune cell wherein the SUV39H1 gene is inactivated or inhibited, wherein said cell expresses an antigen-specific receptor that specifically binds to an antigen. The antigen-specific receptor may be a chimeric antigen receptor (CAR) comprising: a) an extracellular antigen-binding domain, b) a transmembrane domain, c) optionally one or more costimulatory domains, and d) an intracellular signaling domain comprising an intracellular signaling domain with a single active ITAM domain, e.g. a modified CD3zeta domain in which ITAM2 and ITAM3 have been inactivated. This can be accomplished by any means known in the art, e.g., ITAM2 and ITAM3 have been inactivated, or ITAM1 and ITAM2 have been inactivated. For example, a modified CD3 zeta polypeptide retains only ITAM1 and the remaining CD37 domain is deleted (residues 90-164). As another example, ITAM1 is substituted with the amino acid sequence of ITAM3, and the remaining CD33 domain is deleted (residues 90-164). The antigen-specific receptor may be a TCR comprising an intracellular signaling domain with a single active ITAM domain as described. Examples of antigens to which such CAR or TCR binds, preferably with a binding affinity KD of 10-7 M or 10-8 M or less, include orphan tyrosine kinase receptor ROR1, tEGFR, Her2, p95HER2, LI-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, BCMA, Lewis Y, MAGE-A1, mesothelin, MUC1, MUC16, PSCA, NKG2D Ligands, NY-ESO-1, MART-1, gplOO, oncofetal antigen, TAG72, VEGF-R2, carcinoembryonic antigen (CEA), prostate specific antigen (PSMA), estrogen receptor, progesterone receptor, ephrinB2, CD 123, CS-1, c-Met, GD-2, MAGE A3, CE7, or Wilms Tumor 1 (WT-1).

In any of the aspects described herein, the modified immune cell may be a T cell, a T cell progenitor, a hematopoietic stem cell, an iPSC, a CD4+ T cell, a CD8+ T cell, a CD4+ and CD8+ T cell, or a NK cell, or a TN cell, TSCM, TCM or TEM cell. The modified immune cell may be a T regulatory cell. In any of the aspects described herein, SUV39H1 activity gene may be inhibited by inactivation or disruption of the SUV39H1 gene of the immune cell, or it may be inhibited by expression or delivery of a SUV39H1 inhibitor. In some embodiments, the immune cell retains its wild type gene but is modified to comprise a nucleic acid encoding a SUV39H1 inhibitor, optionally a dominant negative SUV39H1 gene.

In any of the aspects described herein, the antigen-specific receptor is a CAR comprising: (a) an extracellular antigen-binding domain; (b) a transmembrane domain, (c) optionally one or more costimulatory domains, and (d) an intracellular signaling domain. The extracellular antigen-binding domain may be a scFv, optionally an scFv that specifically binds a cancer antigen as disclosed herein. The transmembrane domain may be from CD28, CD8 or CD3-zeta. The one or more costimulatory domains may be 4-1BB, CD28, ICOS, OX40 and/or DAP10. The intracellular signaling domain may comprise the intracellular signaling domain of a CD3-zeta polypeptide, or a fragment thereof, optionally a CD3-zeta polypeptide wherein immunoreceptor tyrosine-based activation motif 2 (ITAM2) and immunoreceptor tyrosine-based activation motif 3 (ITAM3) are inactivated.

In any of these embodiments, the antigen-specific receptor may be a bispecific antigen-specific receptor that binds both (a) a first antigen (e.g. a cancer antigen) and (b) a T cell activation antigen, e.g. CD3 epsilon or the constant chain (alpha or beta) of a TCR.

In any of these embodiments, the immune cell may further comprise a second antigen-specific receptor, optionally a TCR or CAR, that specifically binds to a second antigen. For example, the immune cell may comprise two CARs, a first CAR that binds a first antigen and a second CAR that binds a second antigen.

In any of these embodiments, inactivation of SUV39H1 reduces SUV39H1 expression by at least about 50%, 60%, 70%, 75%, 80%, 85%, 90% or 95%.

In any of these embodiments, the immune cell may be autologous or allogeneic. In any of these embodiments, the immune cell is modified such that the HLA-A locus is inactivated. In some embodiments, HLA class I expression is reduced by at least about 75%, 80%, 85%, 90% or 95%.

The disclosure also provides, in another aspect, a sterile pharmaceutical composition comprising any of the foregoing modified immune cells. The disclosure also provides a kit comprising any of the foregoing modified immune cells and a delivery device or container.

The disclosure further provides a method of using the foregoing modified immune cell or pharmaceutical composition or kit to treat a patient suffering from or at risk of disease associated with the antigen, optionally cancer, by administering a therapeutically effective amount of said immune cell or pharmaceutical composition to the patient. In some embodiments, the immune cell is a CAR T-cell and a dose of less than about 5×107 cells, optionally about 105 to about 107 cells, is administered to the patient. The method may further comprise administering to the patient a second therapeutic agent, optionally one or more cancer chemotherapeutic agents, cytotoxic agents, hormones, anti-angiogens, radiolabelled compounds, immunotherapy, surgery, cryotherapy, and/or radiotherapy, is administered to the patient. The second therapeutic agent may be an immune checkpoint modulator. Examples of an immune checkpoint modulator include an antibody that specifically binds to, or an inhibitor of, PD1, PDL1, CTLA4, LAG3, BTLA, OX2R, TIM-3, TIGIT, LAIR-1, PGE2 receptor, EP2/4 adenosine receptor, or A2AR.

### BRIEF DESCRIPTION OF THE DRAWINGS

FIG. 1 shows SUV39H1 expression levels by RT-qPCR in CD8+ T cells in which SUV39H1 has been knocked out.

FIGS. 2A-2C show fold change of geometric mean fluorescence intensity (MFI) compared to Mock by flow cytometry, indicating increased expression levels of central memory T cell surface markers CCR7, CD27 and CD62L in SUV39H1 knockout cells.

FIG. 3A shows a representative FACS plot indicating expression levels of central memory T cell surface markers CCR7, CD45RO, CD27, and CD62L by flow cytometry in SUV39H1 knockout cells. FIG. 3B shows fold change frequency of the Central Memory Cell subset of CCR7+CD45RO+CD27+CD62L+ cells.

FIG. 4A shows the fold change in frequency of subsets of cells that are (a) TIM-3 positive, PD-1 negative, (b) TIM-3 positive, PD-1 positive, (c) TIM-3 negative, PD-1 positive, (d) TIM-3 negative, PD-1 negative. FIG. 4B shows expression levels of TIM-3 (fold change in mean fluorescence intensity).

FIG. 5A shows expression levels of T-bet (fold change in mean fluorescence intensity). FIG. 5B shows a representative FACS plot indicating expression levels of EOMES and T-bet. FIG. 5C shows the fold change in frequency of subsets of cells that are (a) EOMES positive, Tbet negative, (b) EOMES positive, Tbet positive, (c) EOMES negative, Tbet positive, and (d) EOMES negative, Tbet negative, by flow cytometry. FIG. 5D shows a representative FACS plot indicating expression levels of TCF-1 and T-bet. FIG. 5E shows the fold change in frequency of subsets of cells that are (a) TCF1 positive, Tbet negative, (b) TCF1 positive, Tbet positive, (c) TCF1 negative, Tbet positive, and (d) TCF1 negative, Tbet negative, by flow cytometry. These expression patterns of T cell master transcription factors, T-bet, EOMES and TCF-1 indicate a decreased effector-like phenotype for SUV39H1 knockout cells.

FIG. 6 shows fold change in numbers of CD8+ T-cells each week following serial stimulations, indicating increased proliferation for the SUV39H1 knockout cells.

FIG. 7A shows the percentage of T cells expressing CAR after lentiviral transduction of a second generation anti-CD19 CAR. FIG. 7B shows the specific killing of CD19-positive Raji cells by anti-CD19 CAR T cells measured as change in cell impedance (Cell Index) by the xCelligence device. FIG. 7C shows the specific in vivo anti-tumor activity of the anti-CD19 CAR-T cells in NSG mice injected with luciferase-expressing CD19-positive NALM-6 cells measured as change in bioluminescence.

FIGS. 8A and 8B show the percentage of anti-CD19 CAR-expressing T cells after knockout of SUV39H1 by electroporation with Cas9 RNPs containing SUV39-targeting gRNAs and show the decreased expression levels of SUV39H1 in such cells by RT-qPCR. FIG. 8C shows the expression of CAR in total CD3+ Mock and SUV39H1KO T cells and also within the CD4+ and CD8+ subsets. In FIG. 8D, the amount of trimethylated lysine 9 of histone 3 (H3K9me3) is quantified by flow cytometry. This confirms that inactivation of SUV39H1 has a direct effect on the levels of its substrate, H3K9me3.

FIG. 9A shows the percentage of the anti-CD19 CAR-T cells with SUV39H1 knocked out that exhibit markers of the Central Memory Cell subset CCR7+ CD45RO+ CD27+ CD62L+ by flow cytometry. FIG. 9B shows the fold change in the Central Memory Cell subset frequency.

FIG. 10A shows expression levels of TIM-3 (fold change in mean fluorescence intensity) and FIG. 10B shows expression levels of T-bet (fold change in mean fluorescence intensity) in anti-CD19 CAR-T cells with SUV39H1 knocked out compared to Mock. FIG. 10C shows the fold change in frequency of subsets of SUV39H1KO CAR T cells that are (a) EOMES positive, Tbet negative, (b) EOMES positive, Tbet positive, (c) EOMES negative, Tbet positive, and (d) EOMES negative, Tbet negative, by flow cytometry.

FIG. 11 shows the fold change in numbers of SUV39H1KO CD8+ CAR T cells each week following serial stimulations, for two representative donors, indicating increased proliferation for the SUV39H1KO CAR T cells.

FIG. 12 shows the fold change in numbers of SUV39H1KO CD3+ CAR T cells. Inactivation of SUV39H1 results in increased proliferation compared to Mock.

FIG. 13 shows the expression levels of cytokine signaling and stemness/memory genes of CD8+ CAR T cells (Mock) and of SUV39H1KO CAR T cells.

FIGS. 14A and 14B show respectively the expression levels of glycolysis and effector cytokine genes in CD8+ CAR T cells (Mock) and SUV39H1KO CAR T cells after one week of stimulation. Anti-CD19 CAR-T cells with SUV39H1 knocked out show decreased effector differentiation. FIG. 14C shows the expression levels of genes associated with memory/stemness. SUV39H1KO CAR T cells show increased expression levels of memory/stemness genes.

FIG. 15A shows the expression levels of additional stemness/memory genes. FIG. 15B shows the expression levels of genes associated with terminal effector differentiation (effector cytokines and natural killer (NK) cell receptors). FIG. 15C shows expression levels of exhaustion associated genes. SUV39H1KO CAR T cells display increased expression of stemness/memory genes and decreased expression of terminal effector and exhaustion genes, consistent with the effect of SUV39H1 inactivation in inhibiting terminal differentiation.

FIG. 16 shows the specific in vitro killing of CD19-positive NALM-6 cells by anti-CD19 CAR T cells, either Mock or SUV39H1KO, measured by bioluminescence at a 2:1 effector:target ratio.

FIG. 17A shows the aerobic glycolysis of CAR T cells, either Mock or SUV39H1KO, measured as change in extracellular acidification rate (ECAR) by the extracellular flux analyzer Seahorse (Agilent). FIG. 17B shows the Glycolytic reserve of CAR T cells, either Mock or SUV39H1KO. Inactivation of SUV39H1 marginally increases the glycolytic reserve of CAR T cells.

FIG. 18A shows the mitochondrial respiration of CAR T cells, either Mock or SUV39H1KO, measured as change in oxygen consumption rate (OCR) by the extracellular flux analyzer Seahorse (Agilent). FIG. 18B shows the ATP production of CAR T cells, either Mock or SUV39H1KO. Inactivation of SUV39H1 increases mitochondrial respiration in the absence of glucose and pyruvate and overall ATP production.

FIG. 19A shows the experimental procedure for a xenogeneic tumor model for acute lymphoblastic leukemia. Briefly, 2.5×105 NALM-6 cells expressing luciferase were injected intravenously in the tail of NSG mice and their growth in vivo was followed longitudinally by bioluminescence (IVIS, Perkin Elmer). On Day 3 post tumor injection, we infused 106 CAR T cells, either Mock or SUV39H1KO. FIG. 19B shows the growth of NALM-6 cells and the Kaplan-Meyer survival graphs of NSG mice treated either with Mock or SUV39H1KO CAR T cells. SUV39H1KO CAR T cells displayed stronger anti-tumor response and enhanced the survival of NSG mice.

FIG. 20A shows the growth of NALM-6 cells in NSG mice treated with 2×106 CAR T cells, either Mock or SUV39H1KO. FIG. 20B shows the Kaplan-Meyer survival graph of NSG mice treated either with 2×106 CAR T cells, either Mock or SUV39H1KO. SUV39H1 CAR T cells displayed stronger anti-tumor response and enhanced the survival of NSG mice (9 out of 10).

FIG. 21A shows the experimental procedure combining in frame knock-in/knock-out for CAR insertion in the TRAC locus and parallel knock-out of SUV39H1. FIG. 21B shows the percentage of CAR expressing cells and the mean fluorescence intensity of CAR+ cells (quantifying the number of CAR molecules at the cell surface) in the CD4+ and CD8+ subsets. FIG. 21C shows the expression levels of SUV39H1 in CAR T cells treated with gRNAs for TRAC only or TRAC and SUV39H1 (shown as “SUV” in the figure).

### DETAILED DESCRIPTION

### Definitions

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody) fragments. The term encompasses recombinant and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgG1, IgG2, IgG3, IgG4, IgM, IgE, IgA, and IgD. In some embodiments the antibody comprises a heavy chain variable region and a light chain variable region.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; variable heavy chain (VH) regions, VHH antibodies, single-chain antibody molecules such as scFvs and single-domain VH single antibodies; and multispecific antibodies formed from antibody fragments. In particular embodiments, the antibodies are single-chain antibody fragments comprising a variable heavy chain region and/or a variable light chain region, such as scFvs.

“Single-domain antibodies” are antibody fragments comprising all or a portion of the heavy chain variable domain or all or a portion of the light chain variable domain of an antibody. In certain embodiments, a single-domain antibody is a human single-domain antibody.

“Inactivation” or “disruption” of a gene refers to a change in the sequence of genomic DNA that causes the gene's expression to be reduced or eliminated, or that cause a non-functional gene product to be expressed. Exemplary methods include gene silencing, knockdown, knockout, and/or gene disruption techniques, such as gene editing through, e.g., induction of breaks and/or homologous recombination. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

“Inhibition” of a gene product refers to a decrease of its activity and/or gene expression of at least 30%, 40%, 50%, 60%, 70%, 80%, 90%, 95% or 99% or more as compared to the activity or expression levels of wildtype which is not inhibited or repressed.

“Non-functional” refers to a protein with reduced activity or a lack of detectable activity compared to wildtype protein.

“Dominant negative” gene product refers to a mutated non-functional gene product that interferes with or adversely affects the function of the wildtype product within the same cell. Typically, the ability of the mutated gene product to interact with the same elements as the wildtype product remains, but some functional aspects are blocked.

### Cells of the Invention

### Immune Cells

The immune cells according to the invention are typically mammalian cells, e.g., human cells.

More particularly, the cells of the invention are derived from the blood, bone marrow, lymph, or lymphoid organs (notably the thymus) and are cells of the immune system (i.e., immune cells), such as cells of the innate or adaptive immunity, e.g., myeloid or lymphoid cells, including lymphocytes, typically T cells and/or NK cells. Preferably according to the invention, cells are notably lymphocytes including T cells, B cells and NK cells.

Cells according to the invention may also be immune cell progenitors, such as lymphoid progenitors and more preferably T cell progenitors. Examples of T-cell progenitors include induced pluripotent stem cells (iPSCs), hematopoietic stem cells (HSCs), multipotent progenitor (MPP);lymphoid-primed multipotent progenitor (LMPP); common lymphoid progenitor (CLP); lymphoid progenitor (LP); thymus settling progenitor (TSP); early thymic progenitor (ETP). Hematopoietic stem and progenitor cells can be obtained, for example, from cord blood, or from peripheral blood, e.g. peripheral blood-derived CD34+ cells after mobilization treatment with granulocyte-colony stimulating factor (G-CSF).

T cell progenitors typically express a set of consensus markers including CD44, CD117, CD135, and Sca-1 but see also Petrie HT, Kincade PW. Many roads, one destination for T cell progenitors. The Journal of Experimental Medicine. 2005;202(1):11-13.

The cells typically are primary cells, such as those isolated directly from a subject and/or isolated from a subject and frozen.

With reference to the subject to be treated, the cells of the invention may be allogeneic and/or autologous.

In autologous immune cell therapy, immune cells are collected from the patient, modified as described herein, and returned to the patient. In allogeneic immune cell therapy, immune cells are collected from healthy donors, rather than the patient, modified as described herein, and administered to patients. Typically these are HLA matched to reduce the likelihood of rejection by the host. The immune cells may also comprise modifications such as disruption or removal of HLA class I molecules. For example, Torikai et al., Blood. 2013; 122:1341-1349 used ZFNs to knock out the HLA-A locus, while Ren et al., Clin. Cancer Res. 2017; 23:2255-2266 knocked out Beta-2 microglobulin (B2M), which is required for HLA class I expression.

In addition, universal ‘off the shelf’ product immune cells must comprise modifications designed to reduce graft vs. host disease, such as inactivation (e.g. disruption or deletion) of the TCRαβ receptor; the resulting cell exhibits significantly reduced or nearly eliminated expression of the endogenous TCR. See Graham et al., Cells. 2018 Oct; 7 (10): 155 for a review. Because a single gene encodes the alpha chain (TRAC) rather than the two genes encoding the beta chain, the TRAC locus is a typical target for removing or disrupting TCRαβ receptor expression, although the TCRβ loci may alternatively be disrupted. Alternatively, inhibitors of TCRαβ signaling may be expressed, e.g. truncated forms of CD3ζ can act as a TCR inhibitory molecule. Ren et al. simultaneously knocked out TCRαβ, B2M and the immune-checkpoint PD1.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen-specific receptor, presence in a particular organ or compartment, marker or cytokine secretion profile, and/or degree of differentiation.

Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. Preferably, the cells according to the invention are TEFF cells with stem/memory properties and higher reconstitution capacity due to the inhibition of SUV39H1, as well as TN cells, TSCM, TCM, TEM cells and combinations thereof.

In some embodiments, one or more of the T cell populations is enriched for, or depleted of, cells that are positive for or express high levels of one or more particular markers, such as surface markers, or that are negative for or express relatively low levels of one or more markers. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD117, CD135, CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8+ T cells are enriched for cells positive for CD45RO (or negative for CD45RA) and for CD62L. The subset of cells that are CCR7+, CD45RO+, CD27+, CD62L+ cells constitute a central memory cell subset.

For example, according to the invention, the cells can include a CD4+ T cell population and/or a CD8+ T cell sub-population, e.g., a sub-population enriched for central memory (TCM) cells. Alternatively, the cells can be other types of lymphocytes, including natural killer (NK) cells, mucosal associated invariant T (MAIT) cells, Innate Lymphoid Cells (ILCs) and B cells.

The cells and compositions containing the cells for engineering according to the invention are isolated from a sample, notably a biological sample, e.g., obtained from or derived from a subject. Typically, the subject is in need for a cell therapy (adoptive cell therapy) and/or is the one who will receive the cell therapy. The subject is preferably a mammal, notably a human. In one embodiment of the invention, the subject has a cancer.

The samples include tissue, fluid, and other samples taken directly from the subject, as well as samples resulting from one or more processing steps, such as separation, centrifugation, genetic engineering (for example transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom. Preferably, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, and/or cells derived therefrom. Samples include, in the context of cell therapy (typically adoptive cell therapy) samples from autologous and allogeneic sources.

In some embodiments, the cells are derived from cell lines, e.g., T cell lines. The cells can also be obtained from a xenogeneic source, such as a mouse, a rat, a non-human primate, or a pig. Preferably, the cells are human cells.

SUV39H1 Human SUV39H1 methyltransferase is referenced as O43463 in UNIPROT and is encoded by the gene SUV39H1 located on chromosome x (gene ID: 6839 in NCBI). One exemplary human gene sequence is SEQ ID NO: 1, and one exemplary human protein sequence is SEQ ID NO: 2, but it is understood that polymorphisms or variants with different sequences exist in various subjects' genomes. The term SUV39H1 according to the invention thus encompasses all mammalian variants of SUV39H1, and genes that encode a protein at least 75%, 80%, or typically 85%, 90%, or 95% identical to SEQ ID NO: 2 that has SUV39H1 activity (i.e., the methylation of Lys-9 of histone H3 by H3K9-histone methyltransferase).

“Reduced expression of SUV39H1” as per the invention refers to a decrease of SUV39H1 expression of at least 30%, 40%, 50%, 60%, 70%, 80%, 90%, 95% or 99% or more as compared to normal levels.

By “non-functional” SUV39H1 protein it is herein intended a protein with a reduced activity or a lack of detectable activity as described above.

As used herein, the expression “percentage of identity” between two sequences, means the percentage of identical bases or amino acids between the two sequences to be compared, obtained with the best alignment of said sequences, this percentage being purely statistical and the differences between these two sequences being randomly spread over the two sequences. As used herein, “best alignment” or “optimal alignment”, means the alignment for which the determined percentage of identity (see below) is the highest. Sequence comparison between two nucleic acids sequences is usually realized by comparing these sequences that have been previously aligned according to the best alignment; this comparison is realized on segments of comparison in order to identify and compared the local regions of similarity. The best sequences alignment to perform comparison can be realized, besides manually, by using the global homology algorithm developed by SMITH and WATERMAN (Ad. App. Math., vol.2, p: 482, 1981), by using the local homology algorithm developed by NEDDLEMAN and WUNSCH (J. Mol. Biol, vol.48, p:443, 1970), by using the method of similarities developed by PEARSON and LIPMAN (Proc. Natl. Acd. Sci. USA, vol.85, p:2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, WI USA), by using the MUSCLE multiple alignment algorithms (Edgar, Robert C, Nucleic Acids Research, vol. 32, p: 1792, 2004). To get the best local alignment, one can preferably use BLAST software. The identity percentage between two sequences is determined by comparing these two sequences optimally aligned, the sequences being able to comprise additions or deletions in respect to the reference sequence in order to get the optimal alignment between these two sequences. The percentage of identity is calculated by determining the number of identical positions between these two sequences, and dividing this number by the total number of compared positions, and by multiplying the result obtained by 100 to get the percentage of identity between these two sequences.

### Antigen-Specific Receptors

In some embodiments, the immune cells express antigen-specific receptors on the surface. The cells thus may comprise one or more nucleic acids that encode one or more antigen-specific receptors, optionally operably linked to a heterologous regulatory control sequence. Typically such antigen-specific receptors bind the target antigen with a Kd binding affinity of 10⁻⁶M or less, 10⁻⁷ M or less, 10⁻⁸ M or less, 10⁻⁹ M or less, 10⁻¹⁰ M or less, or 10⁻¹¹ M or less (lower numbers indicating greater binding affinity).

Typically, the nucleic acids are heterologous, (i.e., for example which are not ordinarily found in the cell being engineered and/or in the organism from which such cell is derived). In some embodiments, the nucleic acids are not naturally occurring, including chimeric combinations of nucleic acids encoding various domains from multiple different cell types. The nucleic acids and their regulatory control sequences are typically heterologous. For example, the nucleic acid encoding the antigen-specific receptor may be heterologous to the immune cell and operatively linked to an endogenous promoter of the T-cell receptor such that its expression is under control of the endogenous promoter. In some embodiments, the nucleic acid encoding a CAR is operatively linked to an endogenous TRAC promoter.

Among the antigen-specific receptors as per the invention are recombinant T cell receptors (TCRs) and components thereof, as well as functional non-TCR antigen-specific receptors, such as chimeric antigen receptors (CAR).

The immune cells, particularly if allogeneic, may be designed to reduce graft vs. host disease, such that the cells comprise inactivated (e.g. disrupted or deleted) TCRαβ receptor. Because a single gene encodes the alpha chain (TRAC) rather than the two genes encoding the beta chain, the TRAC locus is a typical target for reducing TCRαβ receptor expression. Thus, the nucleic acid encoding the antigen-specific receptor (e.g. CAR or TCR) may be integrated into the TRAC locus at a location, preferably in the 5′ region of the first exon (SEQ ID NO: 3), that significantly reduces expression of a functional TCR alpha chain. See, e.g., Jantz et al., WO 2017/062451; Sadelain et al., WO 2017/180989; Torikai et al,. Blood, 119(2): 5697-705 (2012); Eyquem et al., Nature. 2017 Mar 2;543(7643):113-117. Expression of the endogenous TCR alpha may be reduced by at least 50%, 60%, 70%, 75%, 80%, 85%, 90%, 95%, 98%, or 99%. In such embodiments, expression of the nucleic acid encoding the antigen-specific receptor is optionally under control of the endogenous TCR-alpha promoter.

### Chimeric Antigen Receptors (CARs)

In some embodiments, the engineered antigen-specific receptors comprise chimeric antigen receptors (CARs), including activating or stimulatory CARs, costimulatory CARs (see WO2014/055668), and/or inhibitory CARs (iCARs, see Fedorov et al., Sci. Transl. Medicine, 5(215) (December 2013)).

Chimeric antigen receptors (CARs), (also known as Chimeric immunoreceptors, Chimeric T cell receptors, Artificial T cell receptors) are engineered antigen-specific receptors, which graft an arbitrary specificity onto an immune effector cell (T cell). Typically, these receptors are used to graft the specificity of a monoclonal antibody onto a T cell, with transfer of their coding sequence facilitated by retroviral vectors.

CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

The CAR may include
(a) an extracellular antigen-binding domain,
(b) a transmembrane domain,
(c) optionally a co-stimulatory domain, and
(d) an intracellular signaling domain.

In some embodiments, the CAR is constructed with a specificity for a particular antigen (or marker or ligand), such as an antigen expressed in a particular cell type to be targeted by adoptive cell therapy, such as a cancer marker. The CAR typically includes in its extracellular portion one or more antigen binding molecules, such as one or more antigen-binding fragment, domain, or portion of an antibody, typically one or more antibody variable domains. For example, the extracellular antigen-binding domain may comprise a light chain variable domain and a heavy chain variable domain, typically as an scFv.

The moieties used to bind to antigen include three general categories, either single-chain antibody fragments (scFvs) derived from antibodies, Fab's selected from libraries, or natural ligands that engage their cognate receptor (for the first-generation CARs). Successful examples in each of these categories are notably reported in Sadelain M, Brentjens R, Riviere I. The basic principles of chimeric antigen receptor (CAR) design. Cancer discovery. 2013; 3(4):388-398 (see notably table 1) and are included in the present application.

Antibodies include chimeric, humanized or human antibodies, and can be further affinity matured and selected as described above. Chimeric or humanized scFv's derived from rodent immunoglobulins (e.g. mice, rat) are commonly used, as they are easily derived from well-characterized monoclonal antibodies. Humanized antibodies contain rodent-sequence derived CDR regions; typically the rodent CDRs are engrafted into a human framework, and some of the human framework residues may be back-mutated to the original rodent framework residue to preserve affinity, and/or one or a few of the CDR residues may be mutated to increase affinity. Fully human antibodies have no murine sequence, and are typically produced via phage display technologies of human antibody libraries, or immunization of transgenic mice whose native immunoglobin loci have been replaced with segments of human immunoglobulin loci. Variants of the antibodies can be produced that have one or more amino acid substitutions, insertions, or deletions in the native amino acid sequence, wherein the antibody retains or substantially retains its specific binding function. Conservative substitutions of amino acids are well known and described above. Further variants may also be produced that have improved affinity for the antigen.

Typically, the CAR includes an antigen-binding portion or portions of an antibody molecule, such as a single-chain antibody fragment (scFv) derived from the variable heavy (VH) and variable light (VL) chains of a monoclonal antibody (mAb).

In some embodiments, the CAR comprises an antibody heavy chain variable domain that specifically binds the antigen, such as a cancer marker or cell surface antigen of a cell or disease to be targeted, such as a tumor cell or a cancer cell, such as any of the target antigens described herein or known in the art.

In some embodiments, the CAR contains an antibody or an antigen-binding fragment (e.g. scFv) that specifically recognizes an antigen, such as an intact antigen, expressed on the surface of a cell.

In some embodiments, the CAR contains a TCR-like antibody, such as an antibody or an antigen-binding fragment (e.g. scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen-specific receptor. Among the antigen-specific receptors are functional non-TCR antigen-specific receptors, such as chimeric antigen receptors (CARs). Generally, a CAR containing an antibody or antigen-binding fragment that exhibits TCR-like specificity directed against peptide-MHC complexes also may be referred to as a TCR-like CAR.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that is naturally associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain can be derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD 134, CD137, CD154, ICOS or a GITR). The transmembrane domain can also be synthetic. In some embodiments, the transmembrane domain is derived from CD28, CD8 or CD3-zeta.

In some embodiments, a short oligo- or polypeptide linker, for example, a linker of between 2 and 10 amino acids in length, is present and forms a linkage between the transmembrane domain and the cytoplasmic signaling domain of the CAR.

The CAR generally includes at least one intracellular signaling component or components. First generation CARs typically had the intracellular domain from the CD3 ζ-chain, which is the primary transmitter of signals from endogenous TCRs. Second generation CARs typically further comprise intracellular signaling domains from various costimulatory protein receptors (e.g., CD28, 41BB (CD28), ICOS) to the cytoplasmic tail of the CAR to provide additional signals to the T cell. Co-stimulatory domains include domains derived from human CD28, 4-1BB (CD137), ICOS-1, CD27, OX 40 (CD137), DAP10, and GITR (AITR). Combinations of two co-stimulatory domains are contemplated, e.g. CD28 and 4-1BB, or CD28 and OX40. Third generation CARs combine multiple signaling domains, such as CD3z-CD28-4-1BB or CD3z-CD28-OX40, to augment potency.

The intracellular signaling domain can be from an intracellular component of the TCR complex, such as a TCR CD3+ chain that mediates T-cell activation and cytotoxicity, e.g., the CD3 zeta chain. Alternative intracellular signaling domains include FcεRIγ. The intracellular signaling domain may comprise a modified CD3 zeta polypeptide lacking one or two of its three immunoreceptor tyrosine-based activation motifs (ITAMs), wherein the ITAMs are ITAM1, ITAM2 and ITAM3 (numbered from the N-terminus to the C-terminus). The intracellular signaling region of CD3-zeta is residues 22-164 of SEQ ID NO: 4. ITAM1 is located around amino acid residues 61-89, ITAM2 around amino acid residues 100-128, and ITAM3 around residues 131-159. Thus, the modified CD3 zeta polypeptide may have any one of ITAM1, ITAM2, or ITAM3 inactivated. Alternatively, the modified CD3 zeta polypeptide may have any two ITAMs inactivated, e.g. ITAM2 and ITAM3, or ITAM1 and ITAM2. Preferably, ITAM3 is inactivated, e.g. deleted. More preferably, ITAM2 and ITAM3 are inactivated, e.g. deleted, leaving ITAM1. For example, one modified CD3 zeta polypeptide retains only ITAM1 and the remaining CD3ζ domain is deleted (residues 90-164). As another example, ITAM1 is substituted with the amino acid sequence of ITAM3, and the remaining CD3ζ domain is deleted (residues 90-164). See, for example, Bridgeman et al., Clin. Exp. Immunol. 175(2): 258-67 (2014); Zhao et al., J. Immunol. 183(9): 5563-74 (2009); Maus et al., WO 2018/132506; Sadelain et al., WO/2019/133969, Feucht et al., Nat Med. 25(1):82-88 (2019).

Thus, in some aspects, the antigen binding molecule is linked to one or more cell signaling modules. In some embodiments, cell signaling modules include CD3 transmembrane domain, CD3 intracellular signaling domains, and/or other CD transmembrane domains. The CAR can also further include a portion of one or more additional molecules such as Fc receptor γ, CD8, CD4, CD25, or CD16.

In some embodiments, upon ligation of the CAR, the cytoplasmic domain or intracellular signaling domain of the CAR activates at least one of the normal effector functions or responses of the corresponding non-engineered immune cell (typically a T cell). For example, the CAR can induce a function of a T cell such as cytolytic activity or T-helper activity, secretion of cytokines or other factors.

In some embodiments, the intracellular signaling domain(s) include the cytoplasmic sequences of the T cell receptor (TCR), and in some aspects also those of co-receptors that in the natural context act in concert with such receptor to initiate signal transduction following antigen-specific receptor engagement, and/or a variant of such molecules, and/or any synthetic sequence that has the same functional capability.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

The CAR can also include a signaling domain and/or transmembrane portion of a costimulatory receptor, such as CD28, 4-1BB, OX40, DAP10, and ICOS. In some aspects, the same CAR includes both the activating and costimulatory components; alternatively, the activating domain is provided by one CAR whereas the costimulatory component is provided by another CAR recognizing another antigen.

The CAR or other antigen-specific receptor can also be an inhibitory CAR (e.g. iCAR) and includes intracellular components that dampen or suppress a response, such as an immune response. Examples of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell. Such CARs are used, for example, to reduce the likelihood of off-target effects when the antigen recognized by the activating receptor, e.g, CAR, is also expressed, or may also be expressed, on the surface of normal cells.

### TCRs

In some embodiments, the antigen-specific receptors include recombinant T cell receptors (TCRs) and/or TCRs cloned from naturally occurring T cells. Nucleic acid encoding the TCR can be obtained from a variety of sources, such as by polymerase chain reaction (PCR) amplification of naturally occurring TCR DNA sequences, followed by expression of antibody variable regions, followed by selecting for specific binding to antigen. In some embodiments, the TCR is obtained from T-cells isolated from a patient, or from cultured T-cell hybridomas. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

A “T cell receptor” or “TCR” refers to a molecule that contains a variable a and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et ah, Immunobiology: The Immune System in Health and Disease, 3 rd Ed., Current Biology Publications, p. 4:33, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) {see, e.g., Jores et al., Pwc. Nat'lAcad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains {e.g., a-chain, β-chain) can contain two immunoglobulin domains, a variable domain {e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) at the N-terminus, and one constant domain {e.g., α-chain constant domain or Cα, typically amino acids 117 to 259 based on Kabat, β-chain constant domain or Cβ, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the a and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3ζ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3 γ-, δ-, ε- and ζ-chains, together with the TCR, form what is known as the T cell receptor complex.

In some embodiments, the TCR may be a heterodimer of two chains a and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

Exemplary antigen-specific receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen-specific receptors include a CAR as described in U.S. Pat. No. 7,446,190, and those described in International Patent Application Publication No.: WO/2014055668 A1.

### Antigens

Among the antigens targeted by the antigen-specific receptors are those expressed in the context of a disease, condition, or cell type to be targeted via the adoptive cell therapy. Among the diseases and conditions are proliferative, neoplastic, and malignant diseases and disorders, more particularly cancers. Infectious diseases and autoimmune, inflammatory or allergic diseases are also contemplated.

The cancer may be a solid cancer or a “liquid tumor” such as cancers affecting the blood, bone marrow and lymphoid system, also known as tumors of the hematopoietic and lymphoid tissues, which notably include leukemia and lymphoma. Liquid tumors include for example acute myelogenous leukemia (AML), chronic myelogenous leukemia (CML), acute lymphocytic leukemia (ALL), and chronic lymphocytic leukemia (CLL), (including various lymphomas such as mantle cell lymphoma, non-Hodgkins lymphoma (NHL), adenoma, squamous cell carcinoma, laryngeal carcinoma, gallbladder and bile duct cancers, cancers of the retina such as retinoblastoma).

Solid cancers notably include cancers affecting one of the organs selected from the group consisting of colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast, head and neck region, testis, prostate and the thyroid gland.

Preferably, a cancer according to the invention is a cancer affecting the blood, bone marrow and lymphoid system as described above. In some embodiments, the cancer is, or is associated, with multiple myeloma.

Diseases according to the invention also encompass infectious diseases or conditions, such as, but not limited to, viral, retroviral, bacterial, and protozoal infections, HIV immunodeficiency, Cytomegalovirus (CMV), Epstein-Barr virus (EBV), adenovirus, BK polyomavirus.

Diseases according to the invention also encompass autoimmune or inflammatory diseases or conditions, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or diseases or conditions associated with transplant. In such circumstances, a T-regulatory cell may be the cell in which SUV39H1 is knocked out.

In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. In some such embodiments, a multi-targeting and/or gene disruption approach as provided herein is used to improve specificity and/or efficacy.

In some embodiments, the antigen is a universal tumor antigen. The term “universal tumor antigen” refers to an immunogenic molecule, such as a protein, that is, generally, expressed at a higher level in tumor cells than in non-tumor cells and also is expressed in tumors of different origins. In some embodiments, the universal tumor antigen is expressed in more than 30%, 40%, 50%, 60%, 70%, 75%, 80%, 85%, 90% or more of human cancers. In some embodiments, the universal tumor antigen is expressed in at least three, at least four, at least five, at least six, at least seven, at least eight or more different types of tumors. In some cases, the universal tumor antigen may be expressed in non-tumor cells, such as normal cells, but at lower levels than it is expressed in tumor cells. In some cases, the universal tumor antigen is not expressed at all in non-tumor cells, such as not expressed in normal cells. Exemplary universal tumor antigens include, for example, human telomerase reverse transcriptase (hTERT), survivin, mouse double minute 2 homolog (MDM2), cytochrome P450 1B1 (CYP1B), HER2/neu, p95HER2, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (DI). Peptide epitopes of tumor antigens, including universal tumor antigens, are known in the art and, in some aspects, can be used to generate MHC-restricted antigen-specific receptors, such as TCRs or TCR-like CARs (see e.g. published PCT application No. WO2011009173 or WO2012135854 and published U.S. application No. US20140065708).

In some aspects, the antigen is expressed on multiple myeloma, such as CD38, CD138, and/or CS-1. Other exemplary multiple myeloma antigens include CD56, TIM-3, CD33, CD123, and/or CD44. Antibodies or antigen-binding fragments directed against such antigens are known and include, for example, those described in U.S. Pat. No. 8,153,765; 8,603477, 8,008,450; U.S. published application No. US20120189622; and published international PCT application Nos. WO2006099875, WO2009080829 or WO2012092612. In some embodiments, such antibodies or antigen-binding fragments thereof (e.g. scFv) can be used to generate a CAR.

In some embodiments, the antigen may be one that is expressed or upregulated on cancer or tumor cells, but that also may be expressed in an immune cell, such as a resting or activated T cell. For example, in some cases, expression of hTERT, survivin and other universal tumor antigens are reported to be present in lymphocytes, including activated T lymphocytes (see e.g., Weng et al. (1996) J Exp. Med., 183:2471-2479; Hathcock et al. (1998) J Immunol, 160:5702-5706; Liu et al. (1999) Proc. Natl Acad Sci., 96:5147-5152; Turksma et al. (2013) Journal of Translational Medicine, 11:152). Likewise, in some cases, CD38 and other tumor antigens also can be expressed in immune cells, such as T cells, such as upregulated in activated T cells. For example, in some aspects, CD38 is a known T cell activation marker.

In some embodiments, the cancer is, or is associated, with overexpression of HER2 or p95HER2. p95HER2 is a constitutively active C-terminal fragment of HER2 that is produced by an alternative initiation of translation at methionine 611 of the transcript encoding the full-length HER2 receptor. The amino acid sequence of p95HER2 is set forth in SEQ ID NO: 5, and the amino acid sequence of the extracellular domain is MPIWKFPDEEGACQPCPINCTHSCVDKDDKGCPAEQRASPLT (SEQ ID NO: 6).

HER2 or p95HER2 has been reported to be overexpressed in breast cancer, as well as gastric (stomach) cancer, gastroesophageal cancer, esophageal cancer, ovarian cancer, uterine endometrial cancer, cervix cancer, colon cancer, bladder cancer, lung cancer, and head and neck cancers. Patients with cancers that express the p95HER2 fragment have a greater probability of developing metastasis and a worse prognosis than those patients who mainly express the complete form of HER2. Saez et al., Clinical Cancer Research, 12:424-431 (2006).

Antigen-binding domains that can specifically bind p95HER2 compared to HER2 (i.e., bind p95HER2 but do not bind significantly to full length HER2 receptor) are disclosed in Sperinde et al., Clin. Cancer Res. 16, 4226-4235 (2010) and U.S. Patent Pub. No. 2013/0316380, incorporated by reference herein in their entireties. Hybridomas that produce monoclonal antibodies that have antigen-binding domains that can specifically bind p95HER2 compared to HER2 are disclosed in Int'l. Patent Pub. No. WO/2010/000565, and in Parra-Palau et al., Cancer Res. 70, 8537-8546 (2010). An example CAR binds the epitope PIWKFPD of p95HER2 with a binding affinity KD of 10-7 M or less, 10-8 M or less, 10-9 M or less or 10-10 M or less. An example antigen-binding domain that specifically binds p95HER2 and its CDRs are disclosed in SEQ ID NO: 14-19 of U.S. Patent Pub. No. 2018/0118849 (SEQ ID NO: 7-12 herein), and preferably have a binding affinity KD for p95HER2 of 10-7 M or less, 10-8 M or less, 10-9 M or less or 10-10 M or less. Rius Ruiz et al., Sci. Transl. Med. 10, eaat1445 (2018) and U.S. Patent Pub. No. 2018/0118849, incorporated by reference herein in their entireties, describe a T-cell bispecific antibody that specifically binds to the epitope PIWKFPD of p95HER2 and to the CD3 epsilon chain of the TCR. The antibody designated p95HER2-TCB consists of an asymmetric two-armed immunoglobulin G1 (IgG1) that binds monovalently to CD3epsilon and bivalently to p95HER2. The bispecific antibody has monovalent low affinity for CD3epsilon of about 70 to 100 nM which reduces the chances of nonspecific activation, and a higher bivalent affinity for p95HER2 of about 9 nM.

In some embodiments as provided herein, an immune cell, such as a T cell, can be engineered to repress or disrupt the gene encoding the antigen in the immune cell so that the expressed antigen-specific receptor does not specifically bind the antigen in the context of its expression on the immune cell itself. Thus, in some aspects, this may avoid off-target effects, such as binding of the engineered immune cells to themselves, which may reduce the efficacy of the engineered in the immune cells, for example, in connection with adoptive cell therapy.

In some embodiments, such as in the case of an inhibitory CAR, the target is an off-target marker, such as an antigen not expressed on the diseased cell or cell to be targeted, but that is expressed on a normal or non-diseased cell which also expresses a disease-specific target being targeted by an activating or stimulatory receptor in the same engineered cell. Exemplary such antigens are MHC molecules, such as MHC class I molecules, for example, in connection with treating diseases or conditions in which such molecules become downregulated but remain expressed in non-targeted cells.

In some embodiments, the engineered immune cells can contain an antigen-specific receptor that targets one or more other antigens. In some embodiments, the one or more other antigens is a tumor antigen or cancer marker. Other antigen targeted by antigen-specific receptors on the provided immune cells can, in some embodiments, include orphan tyrosine kinase receptor ROR1, tEGFR, Her2, p95HER2, LI-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, LI-cell adhesion molecule, MAGE-A1, mesothelin, MUC1, MUC16, PSCA, NKG2D Ligands, NY-ESO-1, MART-1, gplOO, oncofetal antigen, ROR1, TAG72, VEGF-R2, carcinoembryonic antigen (CEA), prostate specific antigen, PSMA, Her2/neu, p95HER2, estrogen receptor, progesterone receptor, ephrinB2, CD 123, CS-1, c-Met, GD-2, and MAGE A3, CE7, Wilms Tumor 1 (WT-1), a cyclin, such as cyclin AI (CCNA1), and/or biotinylated molecules, and/or molecules expressed by HIV, HCV, HBV or other pathogens.

In some embodiments, the CAR binds a pathogen-specific antigen. In some embodiments, the CAR is specific for viral antigens (such as HIV, HCV, HBV, etc.), bacterial antigens, and/or parasitic antigens.

In some embodiments, the cells of the invention is genetically engineered to express two or more antigen-specific receptors on the cell, each recognizing a different antigen and typically each including a different intracellular signaling component. Such multi-targeting strategies are described, for example, in International Patent Application, Publication No.: WO 2014055668 Al (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

Example antigen-binding receptors include bispecific antibodies that are T-cell activating antibodies which bind not only the desired antigen but also an activating T-cell antigen such as CD3 epsilon.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive cell therapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II: 223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In other embodiments of the invention, the cells, e.g., T cells, are not engineered to express recombinant antigen-specific receptors, but rather include naturally occurring antigen-specific receptors specific for desired antigens, such as tumor-infiltrating lymphocytes and/or T cells cultured in vitro or ex vivo, e.g., during the incubation step(s), to promote expansion of cells having particular antigen specificity. For example, in some embodiments, the cells are produced for adoptive cell therapy by isolation of tumor-specific T cells, e.g. autologous tumor infiltrating lymphocytes (TIL). The direct targeting of human tumors using autologous tumor infiltrating lymphocytes can in some cases mediate tumor regression (see Rosenberg SA, et al. (1988) N Engl J Med. 319: 1676-1680). In some embodiments, lymphocytes are extracted from resected tumors. In some embodiments, such lymphocytes are expanded in vitro. In some embodiments, such lymphocytes are cultured with lymphokines (e.g., IL-2). In some embodiments, such lymphocytes mediate specific lysis of autologous tumor cells but not allogeneic tumor or autologous normal cells.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### Method for Obtaining Cells According to the Invention

“Inhibition of SUV39H1 activity” as used herein refers to a decrease of SUV39H1 activity of at least 30%, 40%, 50%, 60%, 70%, 80%, 90%, 95% or 99% or more as compared to the activity or level of the SUV39H1 protein which is not inhibited. Preferentially, the inhibition of SUV39H1 activity leads to the absence in the cell of substantial detectable activity of SUV39H1.

Inhibition of SUV39H1 activity can be achieved through repression of SUV39H1 gene expression, or through inactivation of the SUV39H1 gene of the cell, or through expression of exogenous inhibitors. For example, repression may reduce expression of SUV39H1 in the cell by at least 50, 60, 70, 80, 90, or 95% as to the same cell produced by the method in the absence of the repression. Gene disruption may also lead to a reduced expression of the SUV39H1 protein or to the expression of a non-functional SUV39H1 protein. Inhibition of SUV39H1 in the immune cell according to the present invention can be permanent and irreversible or transient or reversible. Preferably, SUV39H1 inhibition is permanent and irreversible. Inhibition of SUV39H1 in the cell may be achieved prior to or after injection of the cell in the targeted patient as described below.

In some embodiments, the inhibition of SUV39H1 activity in the engineered immune cell disclosed herein is achieved by delivering or expressing at least one agent that inhibits or blocks the expression and/or activity of SUV39H1, i.e. a “SUV39H1 inhibitor.” Suitable SUV39H1 inhibitors include, for example, agents that hybridize or bind to the SUV39H1 gene or its regulatory elements, such as aptamers that block or inhibit SUV39H1 expression or activity; nucleic acid molecules that block transcription or translation, such as antisense molecules complementary to SUV39H1; RNA interfering agents (such as a small interfering RNA (siRNA), small hairpin RNA (shRNA), microRNA (miRNA), or a piwiRNA (piRNA); ribozymes and combination thereof.

Suitable SUV39H1 inhibitors can also include an exogenous nucleic acid comprising a) an engineered, non-naturally occurring Clustered Regularly Interspaced Short Palindromic Repeats (CRISPR) guide RNA that hybridizes with SUV39H1 genomic nucleic acid sequence and/or b) a nucleotide sequence encoding a CRISPR protein (typically a Type-II Cas9 protein), optionally wherein the cells are transgenic for expressing a Cas9 protein. The agent may also be a Zinc finger protein (ZFN) or a TAL protein. The Cas9 protein, TAL protein and/or ZNF protein are linked directly or indirectly to a repressor and/or inhibitor.

Suitable SUV39H1 inhibitors can also include non-functional SUV39H1. In some embodiments, the wildtype SUV39H1 gene is not inactivated, but rather a SUV39H1 inhibitor is expressed in the cell. In some embodiments the inhibitor is a dominant negative SUV39H1 gene that expresses non-functional gene product at a level that inhibits activity of the wildtype SUV39H1. This may comprise overexpression of the dominant negative SUV39H1 gene.

The inactivation of SUV39H1 in the immune cell and the introduction of an antigen-specific receptor that specifically binds to a target antigen can be carried out simultaneously or sequentially in any order.

Inactivation of SUV39H1 in a cell according to the invention may also be effected via repression or disruption of the SUV39H1 gene, such as by deletion, e.g., deletion of the entire gene, exon, or region, and/or replacement with an exogenous sequence, and/or by mutation, e.g., frameshift or missense mutation, within the gene, typically within an exon of the gene. In some embodiments, the disruption results in a premature stop codon being incorporated into the gene, such that the SUV39H1 protein is not expressed or is non-functional. The disruption is generally carried out at the DNA level. The disruption generally is permanent, irreversible, or not transient.

In some embodiments, the gene inactivation is achieved using gene editing agents such as a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease.

Zinc finger, TALE, and CRISPR system binding domains can be “engineered” to bind to a predetermined nucleotide sequence.

In some embodiments, the DNA-targeting molecule, complex, or combination contains a DNA-binding molecule and one or more additional domain, such as an effector domain to facilitate the repression or disruption of the gene. For example, in some embodiments, the gene disruption is carried out by fusion proteins that comprise DNA-binding proteins and a heterologous regulatory domain or functional fragment thereof.

Typically, the additional domain is a nuclease domain. Thus, in some embodiments, gene disruption is facilitated by gene or genome editing, using engineered proteins, such as nucleases and nuclease-containing complexes or fusion proteins, composed of sequence-specific DNA-binding domains fused to, or complexed with, non-specific DNA-cleavage molecules such as nucleases.

These targeted chimeric nucleases or nuclease-containing complexes carry out precise genetic modifications by inducing targeted double-stranded breaks or single-stranded breaks, stimulating the cellular DNA-repair mechanisms, including error-prone nonhomologous end joining (NHEJ) and homology-directed repair (HDR). In some embodiments the nuclease is an endonuclease, such as a zinc finger nuclease (ZFN), TALE nuclease (TALEN), an RNA-guided endonuclease (RGEN), such as a CRISPR-associated (Cas) protein, or a meganuclease. Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29: 135-136; Boch et al. (2009) Science 326: 1509-1512; Moscou and Bogdanove (2009) Science 326: 1501; Weber et al. (2011) PLOS One 6:eI9722; Li et al. (2011) Nucl. Acids Res. 39:6315-6325; Zhang et al. (2011) Nat. Biotech. 29:149-153; Miller et al. (2011) Nat. Biotech. 29: 143-148; Lin et al. (2014) Nucl. Acids Res. 42:e47). Such genetic strategies can use constitutive expression systems or inducible expression systems according to well-known methods in the art.

### ZFPs and ZFNs; TALs, TALEs, and TALENs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs. See Lloyd et al., Frontiers in Immunology, 4(221), 1-7 (2013).

In some embodiments, the DNA-targeting molecule comprises one or more zinc-finger proteins (ZFPs) or domains thereof that bind to DNA in a sequence-specific manner. A ZFP or domain thereof is a protein or domain within a larger protein, that binds DNA in a sequence-specific manner through one or more zinc fingers regions of amino acid sequence within the binding domain whose structure is stabilized through coordination of a zinc ion. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to the target site of choice. See, for example, Beerli et al. (2002) Nature Biotechnol. 20: 135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type IIS restriction enzyme and one or more zinc finger binding domains, which may or may not be engineered. In some embodiments, the cleavage domain is from the Type IIS restriction endonuclease Fok I. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982.

In some aspects, the ZFNs efficiently generate a double strand break (DSB), for example at a predetermined site in the coding region of the targeted gene (i.e. SUV39H1). Typical targeted gene regions include exons, regions encoding N-terminal regions, first exon, second exon, and promoter or enhancer regions. In some embodiments, transient expression of the ZFNs promotes highly efficient and permanent disruption of the target gene in the engineered cells. In particular, in some embodiments, delivery of the ZFNs results in the permanent disruption of the gene with efficiencies surpassing 50%. Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins. Gaj et al., Trends in Biotechnology, 2013, 31(7), 397-405. In some embodiments, commercially available zinc fingers are used or are custom designed. (See, for example, Sigma-Aldrich catalog numbers CSTZFND, CSTZEN, CTI1-1KT, and PZD0020).

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073. In some embodiments, the molecule is a DNA binding endonuclease, such as a TALE-nuclease (TALEN). In some aspects the TALEN is a fusion protein comprising a DNA-binding domain derived from a TALE and a nuclease catalytic domain to cleave a nucleic acid target sequence. In some embodiments, the TALE DNA-binding domain has been engineered to bind a target sequence within genes that encode the target antigen and/or the immunosuppressive molecule. For example, in some aspects, the TALE DNA-binding domain may target CD38 and/or an adenosine receptor, such as A2AR.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation (Critchlow and Jackson, Trends Biochem Sci. 1998 Oct;23(10):394-8) or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

TALE repeats can be assembled to specifically target the SUV39H1 gene. (Gaj et al., Trends in Biotechnology, 2013, 31(7), 397-405). A library of TALENs targeting 18,740 human protein-coding genes has been constructed (Kim et al., Nature Biotechnology. 31, 251-258 (2013)). Custom-designed TALE arrays are commercially available through Cellectis Bioresearch (Paris, France), Transposagen Biopharmaceuticals (Lexington, KY, USA), and Life Technologies (Grand Island, NY, USA). Specifically, TALENs that target CD38 are commercially available (See Gencopoeia, catalog numbers HTN222870-1, HTN222870-2, and HTN222870-3, available on the World Wide Web at www. genecopoeia.com/product/search/detail.php?prt=26&cid=&key=HTN222870). Exemplary molecules are described, e.g., in U.S. Patent Publication Nos. US 2014/0120622, and 2013/0315884.

In some embodiments the TALENs are introduced as transgenes encoded by one or more plasmid vectors. In some aspects, the plasmid vector can contain a selection marker which provides for identification and/or selection of cells which received said vector.

### RGENs (CRISPR/Cas Systems)

The gene repression can be carried out using one or more DNA-binding nucleic acids, such as disruption via an RNA-guided endonuclease (RGEN), or other form of repression by another RNA-guided effector molecule. For example, in some embodiments, the gene repression can be carried out using clustered regularly interspaced short palindromic repeats (CRISPR) and CRISPR-associated proteins. See Sander and Joung, Nature Biotechnology, 32(4): 347-355.

In general, “CRISPR system” refers collectively to transcripts and other elements involved in the expression of, or directing the activity of, CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

Typically, the CRISPR/Cas nuclease or CRISPR/Cas nuclease system includes a non-coding RNA molecule (guide) RNA, which sequence-specifically binds to DNA, and a CRISPR protein, with nuclease functionality (e.g., two nuclease domains). One or more elements of a CRISPR system can derive from a type I, type II, or type III CRISPR system, such as Cas nuclease. Preferably, the CRISPR protein is a Cas enzyme such as Cas9. Cas enzymes are well-known in the field; for example, the amino acid sequence of S. pyogenes Cas9 protein may be found in the SwissProt database under accession number Q99ZW2.In some embodiments, a Cas nuclease and gRNA are introduced into the cell. In some embodiments, the CRISPR system induces DSBs at the target site, followed by disruptions as discussed herein. In other embodiments, Cas9 variants, deemed “nickases” can be used to nick a single strand at the target site. Paired nickases can also be used, e.g., to improve specificity, each directed by a pair of different gRNAs targeting sequences. In still other embodiments, catalytically inactive Cas9 can be fused to a heterologous effector domain, such as a transcriptional repressor, to affect gene expression.

In general, a CRISPR system is characterized by elements that promote the formation of a CRISPR complex at the site of the target sequence. Typically, in the context of formation of a CRISPR complex, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The target sequence may comprise any polynucleotide, such as DNA or RNA polynucleotides. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation. In some embodiments, the CRISPR enzyme, guide sequence, tracr-mate sequence, and tracr sequence are operably linked to and expressed from the same promoter. In some embodiments, a vector comprises a regulatory element operably linked to an enzyme-coding sequence encoding the CRISPR enzyme, such as a Cas protein.

In some embodiments, a CRISPR enzyme in combination with (and optionally complexed with) a guide sequence is delivered to the cell. Typically, CRISPR/Cas9 technology may be used to knockdown gene expression of SUV39H1 in the engineered cells. For example, Cas9 nuclease and a guide RNA specific to the SUV39H1 gene can be introduced into cells, for example, using lentiviral delivery vectors or any of a number of known delivery method or vehicle for transfer to cells, such as any of a number of known methods or vehicles for delivering Cas9 molecules and guide RNAs (see also below).

### Delivery of Nucleic Acids Encoding the Gene Disrupting Molecules and Complexes

In some embodiments, a nucleic acid encoding the DNA-targeting molecule, complex, or combination, is administered or introduced to the cell. Typically, viral and non-viral based gene transfer methods can be used to introduce nucleic acids encoding components of a CRISPR, ZFP, ZFN, TALE, and/or TALEN system to cells in culture.

In some embodiments, the polypeptides are synthesized in situ in the cell as a result of the introduction of polynucleotides encoding the polypeptides into the cell. In some aspects, the polypeptides could be produced outside the cell and then introduced thereto.

Methods for introducing a polynucleotide construct into animal cells are known and include, as non-limiting examples, stable transformation methods wherein the polynucleotide construct is integrated into the genome of the cell, transient transformation methods wherein the polynucleotide construct is not integrated into the genome of the cell, and virus mediated methods.

In some embodiments, the polynucleotides may be introduced into the cell by for example, recombinant viral vectors (e.g. retroviruses, adenoviruses), liposome and the like. Transient transformation methods include microinjection, electroporation, or particle bombardment. The nucleic acid is administered in the form of an expression vector. Preferably, the expression vector is a retroviral expression vector, an adenoviral expression vector, a DNA plasmid expression vector, or an AAV expression vector.

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386,4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

RNA or DNA viral-based systems include retroviral, lentivirus, adenoviral, adeno-associated and herpes simplex virus vectors for gene transfer.

For a review of gene therapy procedures, see Anderson, Science 256:808-813 (1992); Nabel & Feigner, TIBTECH 11:211-217 (1993); Mitani & Caskey, TIBTECH 11: 162-166 (1993); Dillon. TIBTECH 11: 167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1: 13-26 (1994).

A reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into the cell to encode a gene product which serves as a marker by which to measure the alteration or modification of expression of the gene product.

### Cell preparation

Isolation of the cells includes one or more preparation and/or non-affinity based cell separation steps according to well-known techniques in the field. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some embodiments, the cell preparation includes steps for freezing, e.g., cryopreserving, the cells, either before or after isolation, incubation, and/or engineering. Any of a variety of known freezing solutions and parameters in some aspects may be used.

The incubation steps can comprise culture, incubation, stimulation, activation, expansion and/or propagation.

In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a antigen-specific receptor.

The incubation conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include 1L-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al., J Immunother. 2012; 35(9): 651-660, Terakura et al., Blood. 2012; 1:72-82, and/or Wang et al. J Immunother. 2012,35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

In some aspects, the methods include assessing expression of one or more markers on the surface of the engineered cells or cells being engineered. In one embodiment, the methods include assessing surface expression of one or more target antigen (e.g., antigen recognized by the antigen-specific receptor) sought to be targeted by the adoptive cell therapy, for example, by affinity-based detection methods such as by flow cytometry.

### Vectors and Methods for Cell Genetic Engineering

In some aspects, the genetic engineering involves introduction of a nucleic acid encoding the genetically engineered component or other component for introduction into the cell, such as a component encoding a gene-disruption protein or nucleic acid.

Generally, the engineering of CARs into immune cells (e.g., T cells) requires that the cells be cultured to allow for transduction and expansion. The transduction may utilize a variety of methods, but stable gene transfer is required to enable sustained CAR expression in clonally expanding and persisting engineered cells.

In some embodiments, gene transfer is accomplished by first stimulating cell growth, e.g., T cell growth, proliferation, and/or activation, followed by transduction of the activated cells, and expansion in culture to numbers sufficient for clinical applications.

Various methods for the introduction of genetically engineered components, e.g., antigen-specific receptors, e.g., CARs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr 3.; Carlens et al. (2000) Exp Hematol 28(10): 1137-46; Alonso-Camino et al. (2013) Mol Ther Nucl Acids 2, e93; Park et al., Trends Biotechnol. 2011 November; 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3: 102-109.

Methods of lentiviral transduction are also known. Exemplary methods are described in, e.g., Wang et al. (2012) J. Immunother. 35(9): 689-701; Cooper et al. (2003) Blood. 101: 1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation {see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506:115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the genetically engineered nucleic acids encoding the genetically engineered products are those described, e.g., in international patent application, Publication No.: WO2014055668, and U.S. Pat. No. 7,446,190.

### Composition of the Invention

The present invention also includes compositions containing the cells as described herein and/or produced by the provided methods. Typically, said compositions are pharmaceutical compositions and formulations for administration, preferably sterile compositions and formulations, such as for adoptive cell therapy.

A pharmaceutical composition of the invention generally comprises at least one engineered immune cell of the invention and a sterile pharmaceutically acceptable carrier.

As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can further be incorporated into the compositions. In some aspects, the choice of carrier in the pharmaceutical composition is determined in part by the particular engineered CAR or TCR, vector, or cells expressing the CAR or TCR, as well as by the particular method used to administer the vector or host cells expressing the CAR. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001 to about 2% by weight of the total composition.

A pharmaceutical composition is formulated to be compatible with its intended route of administration.

### Therapeutic Methods

The present invention also relates to the cells as previously defined for their use in adoptive cell therapy (notably adoptive T cell therapy), typically in the treatment of cancer in a subject in need thereof, but also in the treatment of infectious diseases and autoimmune, inflammatory or allergic diseases. Treatment of any of the diseases listed above under the “Antigen” section is contemplated.

Treatment”, or “treating” as used herein, is defined as the application or administration of cells as per the invention or of a composition comprising the cells to a patient in need thereof with the purpose to cure, heal, alleviate, relieve, alter, remedy, ameliorate, improve or affect the disease such as cancer, or any symptom of the disease (e.g., cancer). In particular, the terms “treat' or treatment” refers to reducing or alleviating at least one adverse clinical symptom associated with the disease such as the cancer cancer, e.g., pain, swelling, low blood count etc.

With reference to cancer treatment, the term “treat' or treatment” also refers to slowing or reversing the progression neoplastic uncontrolled cell multiplication, i.e. shrinking existing tumors and/or halting tumor growth. The term “treat' or treatment” also refers to inducing apoptosis in cancer or tumor cells in the subject.

The immune cells, particularly T-cells, in which SUV39H1 has been inactivated, exhibit an enhanced central memory phenotype, enhanced survival and persistence after adoptive transfer, and reduced exhaustion. Their increased efficiency and efficacy may allow them to be dosed at lower levels relative to T-cells that do not have the improvements described herein. Thus, T-cells in which SUV39H1 has been inactivated, which optionally have any of the other features described herein (e.g. expressing a CAR, and/or in which a T cell receptor (TCR) alpha constant region gene is inactivated by the insertion of a nucleic acid sequence encoding a CAR or TCR, and/or in which the CAR comprises a) an extracellular antigen-binding domain, b) a transmembrane domain, c) optionally one or more costimulatory domains, and d) an intracellular signaling domain comprising a modified CD3zeta intracellular signaling domain in which ITAM2 and ITAM3 have been inactivated or deleted and/or in which an HLA-A gene has been inactivated or deleted), may be administered at certain doses. For example, the immune cells (e.g., T cells) in which SUV39H1 has been inactivated may be administered to adults at doses of less than about 108 cells, less than about 5×107 cells, less than about 107 cells, less than about 5×106 cells, less than about 106 cells, less than about 5×105 cells or less than about 105 cells. The dose for pediatric patients may be about 100-fold less. In alternative embodiments, any of the immune cells (e.g. T-cells) described herein may be administered to patients at doses ranging from 105 to 109 cells, or 105 to 108 cells, or 106 to 108 cells.

The subject of the invention (i.e. patient) is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS). In some embodiments of the invention, said subject has a cancer, is at risk of having a cancer, or is in remission of a cancer.

The cancer may be a solid cancer or a “liquid tumor” such as cancers affecting the blood, bone marrow and lymphoid system, also known as tumors of the hematopoietic and lymphoid tissues, which notably include leukemia and lymphoma. Liquid tumors include for example acute myelogenous leukemia (AML), chronic myelogenous leukemia (CML), acute lymphocytic leukemia (ALL), and chronic lymphocytic leukemia (CLL), (including various lymphomas such as mantle cell lymphoma, non-Hodgkins lymphoma (NHL), adenoma, squamous cell carcinoma, laryngeal carcinoma, gallbladder and bile duct cancers, cancers of the retina such as retinoblastoma).

Solid cancers notably include cancers affecting one of the organs selected from the group consisting of colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast, head and neck region, testis, prostate and the thyroid gland.

Preferably, a cancer according to the invention is a cancer affecting the blood, bone marrow and lymphoid system as described above. Typically the cancer is, or is associated with, multiple myeloma.

In some embodiments, the subject is suffering from or is at risk of an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus.

In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or a disease or condition associated with transplant

The present invention also relates to a method of treatment and notably an adoptive cell therapy, preferably an adoptive T cell therapy, comprising the administration to a subject in need thereof of a composition a previously described.

In some embodiments, the cells or compositions are administered to the subject, such as a subject having or at risk for a cancer or any one of the diseases as mentioned above. In some aspects, the methods thereby treat, e.g., ameliorate one or more symptom of, the disease or condition, such as with reference to cancer, by lessening tumor burden in a cancer expressing an antigen recognized by the engineered cell.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject. In some embodiments, HLA matching is less important when the immune cell has been modified to reduce expression of endogenous TCR and HLA class I molecules.

Administration of at least one cell according to the invention to a subject in need thereof may be combined with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cell populations are administered prior to the one or more additional therapeutic agents. In some embodiments, the cell populations are administered after to the one or more additional therapeutic agents.

With reference to cancer treatment, a combined cancer treatment can include but is not limited to cancer chemotherapeutic agents, cytotoxic agents, hormones, anti-angiogens, radiolabelled compounds, immunotherapy, surgery, cryotherapy, and/or radiotherapy.

Conventional cancer chemotherapeutic agents include alkylating agents, antimetabolites, anthracyclines, topoisomerase inhibitors, microtubule inhibitors and B-raf enzyme inhibitors.

Alkylating agents include the nitrogen mustards (such as mechlorethamine, cyclophosphamide, ifosfamide, melphalan and chlorambucil), ethylenamine and methylenamine derivatives (such as altretamine, thiotepa), alkyl sulfonates (such as busulfan), nitrosoureas (such as carmustine, lomustine, estramustine), triazenes (such as dacarbazine, procarbazine, temozolomide), and platinum-containing antineoplastic agents (such as cisplatin, carboplatin, oxaliplatin).

Antimetabolites include 5-fluorouracil (5-FU), 6-mercaptopurine (6-MP), Capecitabine (Xeloda®), Cytarabine (Ara-C®), Floxuridine, Fludarabine, Gemcitabine (Gemzar®), Hydroxyurea, Methotrexate, Pemetrexed (Alimta®).

Anthracyclines include Daunorubicin, Doxorubicin (Adriamycin®), Epirubicin. Idarubicin. Other anti-tumor antibiotics include Actinomycin-D, Bleomycin, Mitomycin-C, Mitoxantrone.

Topoisomerase inhibitors include Topotecan, Irinotecan (CPT-11), Etoposide (VP-16), Teniposide or Mitoxantrone

Microtubule inhibitors include Estramustine, Ixabepilone, the taxanes (such as Paclitaxel, Docetaxel and Cabazitaxel), and the vinca alkaloids (such as Vinblastine, Vincristine, Vinorelbine, Vindesine and Vinflunine)

B-raf enzyme inhibitors include vemurafenib (Zelboraf), dabrafenib (Tafinlar), and encorafenib (Braftovi)

Immunotherapy includes but is not limited to immune checkpoint modulators (i.e. inhibitors and/or agonists), cytokines, immunomodulating monoclonal antibodies, cancer vaccines.

Preferably, administration of cells in an adoptive T cell therapy according to the invention is combined with administration of immune checkpoint modulators. Examples include inhibitors of (e.g. antibodies that bind specifically to and inhibit activity of) PD-1, CTLA4, LAG3, BTLA, OX2R, TIM-3, TIGIT, LAIR-1, PGE2 receptors, and/or EP2/4 Adenosine receptors including A2AR. Preferably, the immune checkpoint modulators comprise anti-PD-1 and/or anti-PDL-1 inhibitors (e.g., anti-PD-1 and/or anti-PDL-1 antibodies).

The present invention also relates to the use of a composition comprising the engineered immune cell as herein described for the manufacture of a medicament for treating a cancer, an infectious disease or condition, an autoimmune disease or condition, or an inflammatory disease or condition in a subject.

### EXAMPLES

### Example 1—Inactivating SUV39H1 in human CD8+ T cells (SUV39H1 Knockout T Cells)

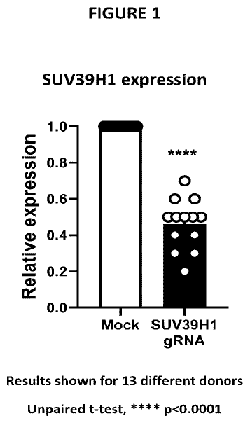

Activated human CD8+ T cells were electroporated with Cas9 ribonucleoprotein particles (RNPs) containing gRNAs that targeted exons of the SUV39H1 gene for deletion. A consistent decrease in SUV39H1 expression was observed by RT-qPCR four days post electroporation, indicating that the knockout was successful (FIG. 1).

### Example 2—Memory Phenotyping of Human SUV39H1KO T Cells

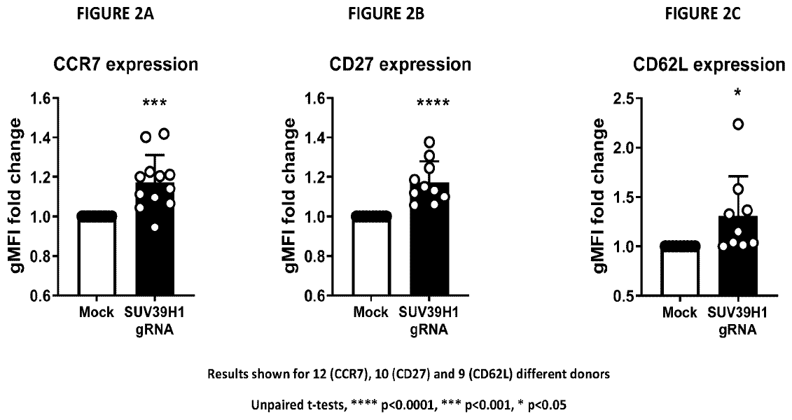

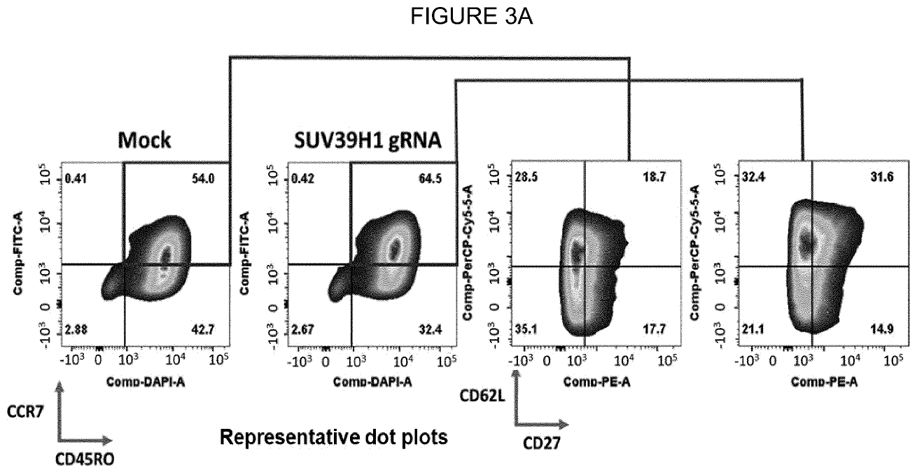

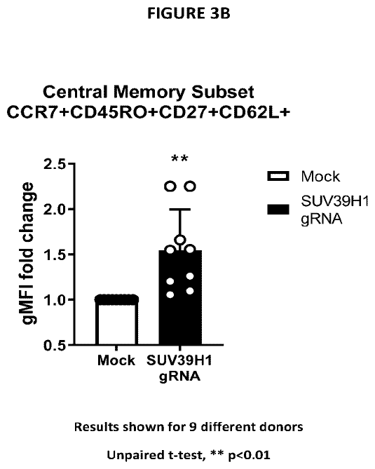

To observe the expression of central memory T cell surface markers important for the memory phenotype of CD8+ T cells, the SUV39H1KO T cells from Example 1 were stimulated with aCD3+aCD28 beads for one week and then analyzed by flow cytometry. The central memory T cell markers CCR7, CD27 and CD62L showed increased levels of expression in SUV39H1KO cells. Results are shown in FIGS. 2A-2C as fold change of geometric MFI for CCR7, CD27 and CD62L, respectively, compared to Mock per donor. Additionally, the fraction of CCR7+CD45RO+CD27+CD62L+ cells, which constitute the central memory cell subset, was increased in SUV39H1 KO cells. Results are shown in FIG. 3A as representative FACS plots and FIG. 3B as fold change frequency of CCR7+CD45RO+CD27+CD62L+ cells compared to Mock per donor. Knocking out SUV39H1 increased the fraction of Central Memory Cells.

### Example 3—Expression of Immune Checkpoint Receptors on Human SUV39H1KO T Cells

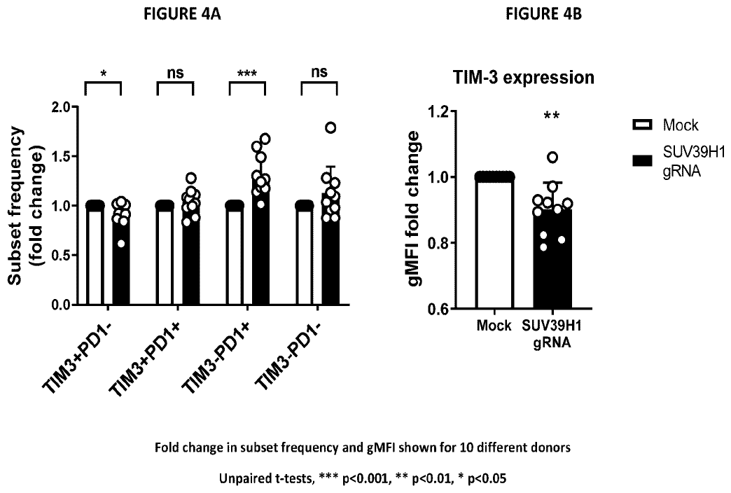

Expression of two important immune checkpoint receptors, PD-1 and TIM-3, was evaluated for the cells of Example 2. The overall expression level of PD-1 was unchanged and the expression level of TIM-3 was decreased. An increase in the fraction of TIM3-PD1+, which are considered as non-exhausted activated cells, and a decrease in the fraction of TIM3+PD1-cells was observed in SUV39H1KO. FIG. 4A shows results of frequency of subsets of cells expressing PD-1 and TIM-3 ((a) TIM-3 pos, PD-1 neg, (b) TIM-3 pos, PD-1 pos, (c) TIM-3 neg, PD-1 pos, (d) TIM-3 neg, PD-1 neg) and FIG. 4B shows fold change of TIM-3 as geometric MFI compared to Mock per donor. Thus, knocking out SUV39H1 reduced T-cell exhaustion.

### Example 4—Expression of Master Transcription Factors, T-Bet, EOMES and TCF-1 on Human SUV39H1KO T Cells

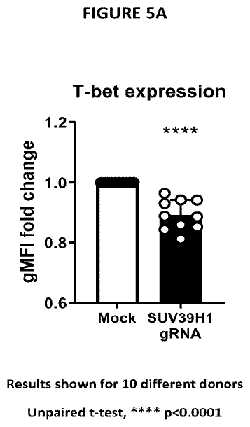

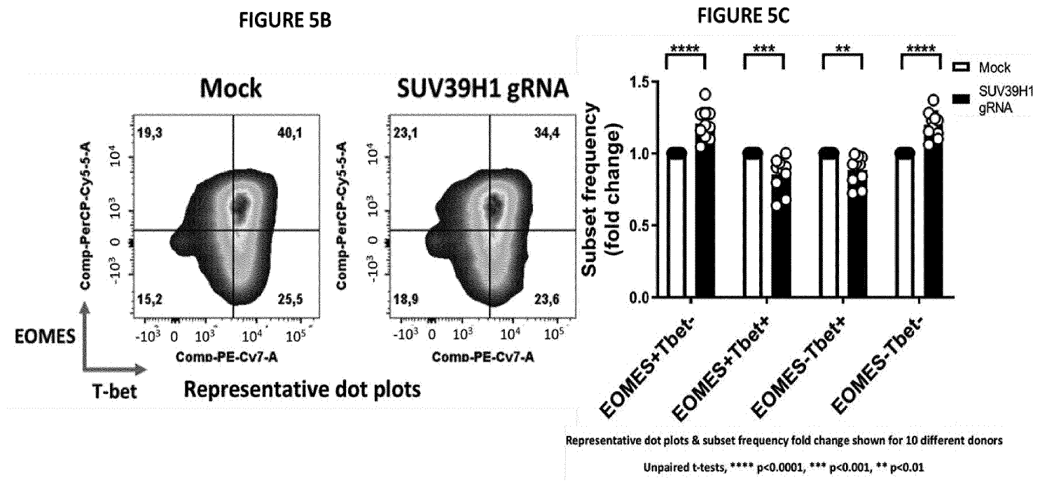

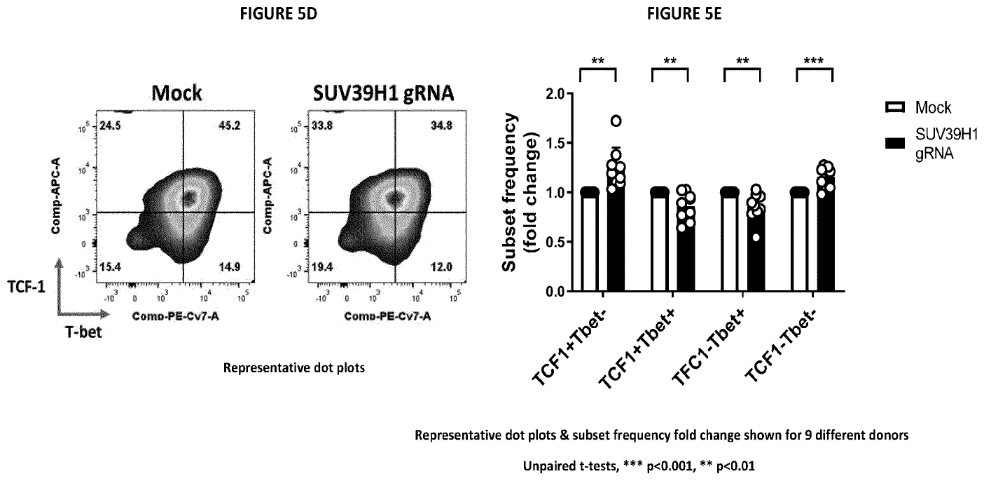

Expression of master transcription factors, T-bet, EOMES and TCF-1 were evaluated for the cells of Example 2. T-bet expression orchestrates increased effector commitment and function, EOMES expression defines increased effector function and TCF-1 expression controls self-renewal. The balance of EOMES and TCF-1 with T-bet determines T cell differentiation. SUV39H1KO resulted in decreased expression levels of T-bet as shown in FIG. 5A as fold change of geometric MFI compared to Mock per donor. The expression levels of EOMES and TCF-1 were unchanged. The balance of T-bet with either EOMES or TCF-1 was also analyzed. EOMES-positive/T-bet-negative and TCF-1-positive/T-bet-negative fractions were increased in SUV39H1KO (FIGS. 5B-5E). The results are shown in FIGS. 5C and 5E as frequency of various subsets of cells compared to Mock per donor and suggested a decrease in effector-like phenotype of SUV39H1KO. Representative FACS plots are shown in FIGS. 5B and 5D.

### Example 5—Proliferation of Human SUV39H1KO T Cells Following Serial Stimulations

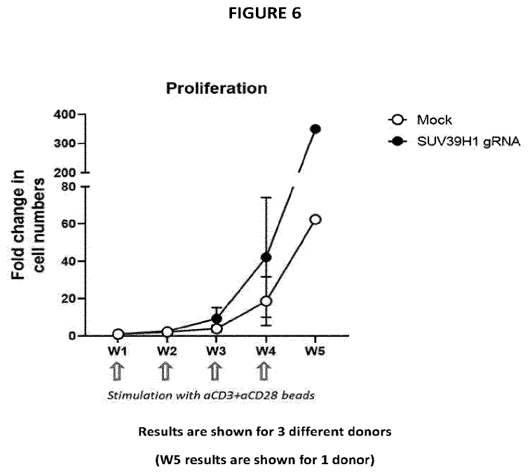

CD8+ T cells in which SUV39H1 was knocked out were stimulated with aCD3+aCD28 beads once a week for a duration of 4 weeks. The cell numbers and kinetics of SUV39H1KO CD8+T cells are depicted in FIG. 6 and show that SUV39H1KO cells displayed increased proliferation after serial stimulations. The results are shown for three different donors as the fold change in number of cells compared to seeding at Week 1. Proliferation potential is a key feature of memory cells and important predictor of anti-tumor efficacy.

In summary, the results of Examples 1-5 show that knocking out SUV39H1 in CD8+ T cells resulted in a) overall increased expression levels of central memory markers, CCR7, CD27 and CD62L, and increased fraction of a Central Memory Cell subset, CCR7+CD45RO+CD27+CD62L+ cells; b) decreased expression levels of TIM-3, c) decreased expression levels of effector function regulator, T-bet, and increased fractions of T-bet negative cells tipping the balance of transcription factors towards a less effector-like phenotype, and d) increased proliferation following serial stimulations.

### Example 6—Generation of CAR T Cells

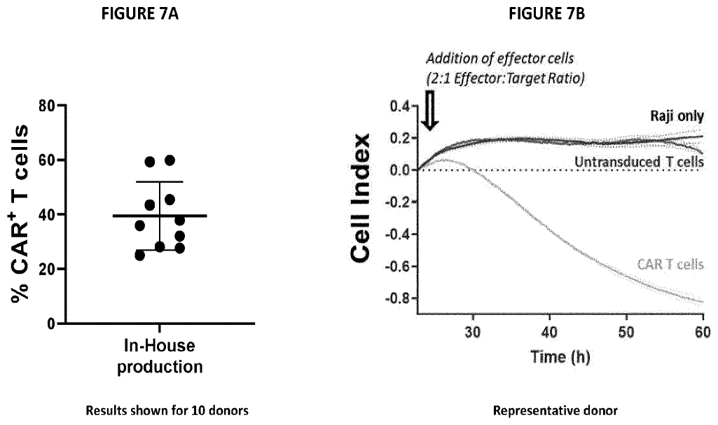

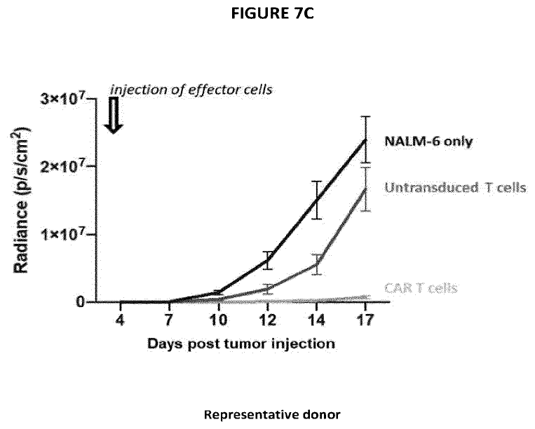

Human CD8+ T cells were transduced with a lentiviral vector containing a gene encoding a second generation anti-CD19 CAR. FIG. 7A shows the percentage of CAR-expressing T cells for 10 donors. FIG. 7B shows killing of CD19-positive Raji cells in the xCelligence device at a 2:1 effector:target ratio for a representative donor. FIG. 7C shows growth of NALM-6 cells, shown as tumor cell bioluminescence intensity, following injection of 5×10⁵ cells in NSG mice after infusion (on day 4) of 6×10⁶ CAR T cells from a representative donor. The results showed that the CAR T cells displayed cytotoxic activity against CD19-positive Raji cells in vitro and also eradicated CD19-positive NALM-6 cells in NSG mice.

### Example 7—Inactivating SUV39H1 in CAR T-Cells (SUV39H1KO CAR T Cells)

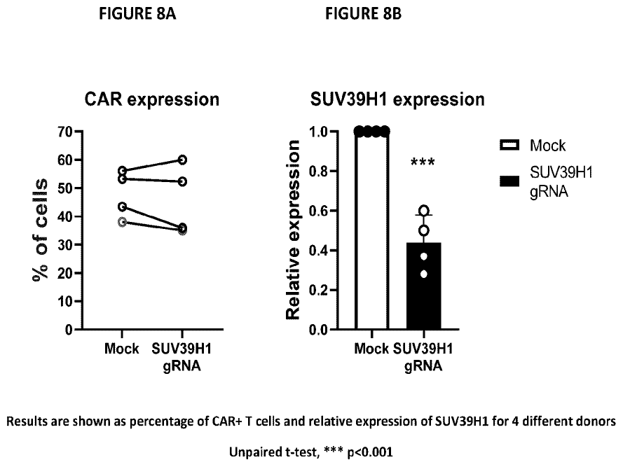

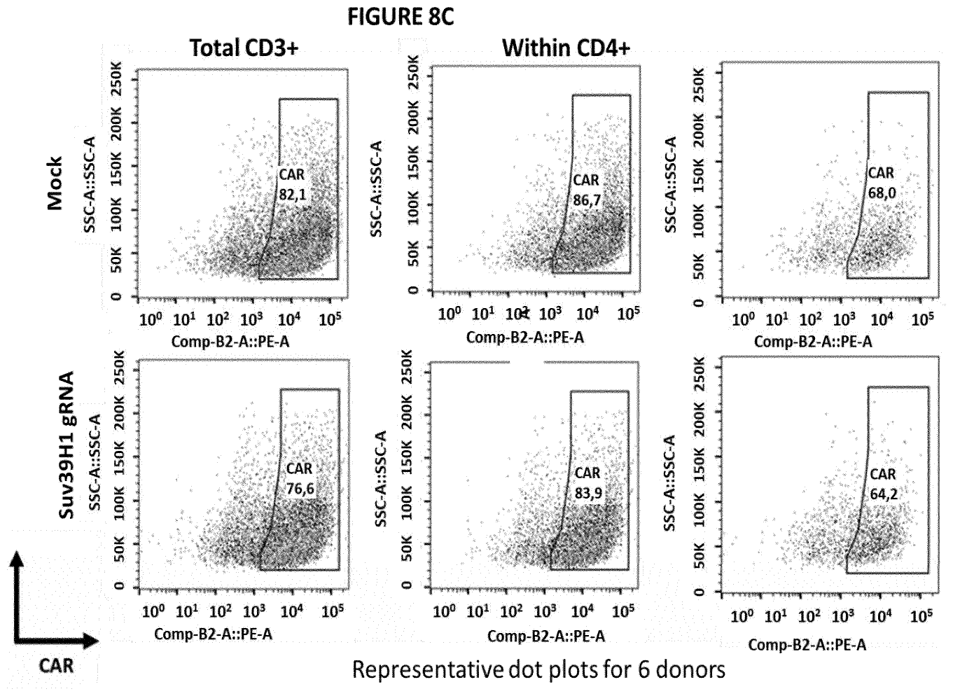

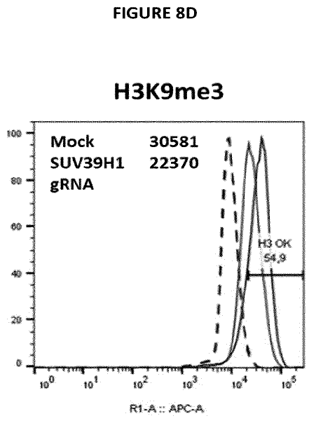

Total CD3+ or CD8+ T cells were purified from PBMCs and lentivirally transduced with the CAR transgene as described in Example 6 that had been lentivirally transduced with the CAR transgene. The cells were then electroporated with Cas9 RNPs containing SUV39H1-targeting gRNAs that targeted exons of the SUV39H1 gene for deletion. The knockout cells (SUV39H1KO T cells) retained and showed robust CAR expression and exhibited a consistent decrease in SUV39H1 expression as detected by RT-qPCR four days post electroporation (FIGS. 8A-8B). Specifically, FIGS. 8A and 8C depict CAR expression of CD8+ and CD3+ T cells, respectively, while FIG. 8B depicts the deletion of SUV39H1 expression. Western blotting further confirmed the depletion of SUV39H1 protein in FIG. 8B, and that the levels of H3K9me3, which are dependent on SUV39H1 activity, are globally decreased in SUV39H1KO T cells (FIG. 8D).

### Example 8—Memory Phenotype, Master Transcription Factor Expression, Gene Expression Profiles, and Proliferation of SUV39H1KO CAR T Cells

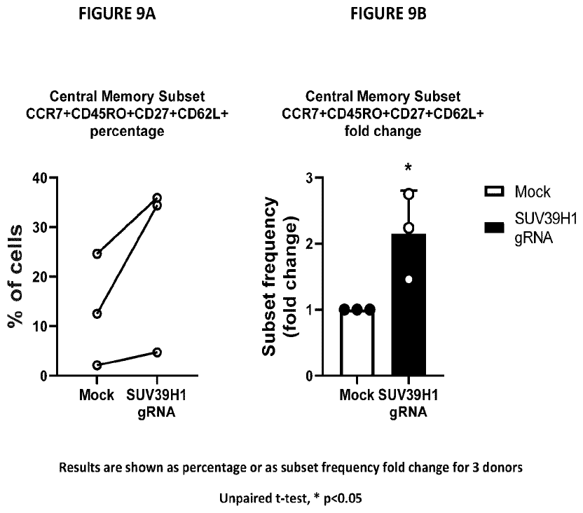

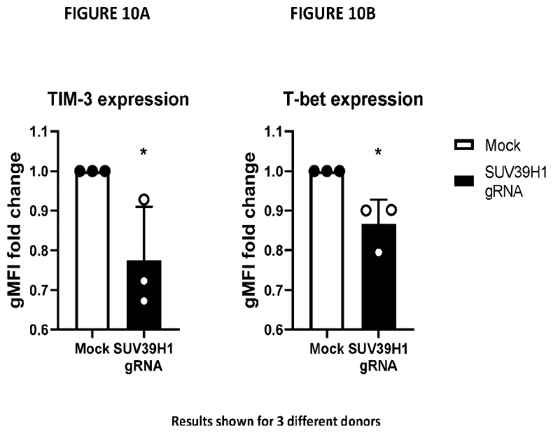

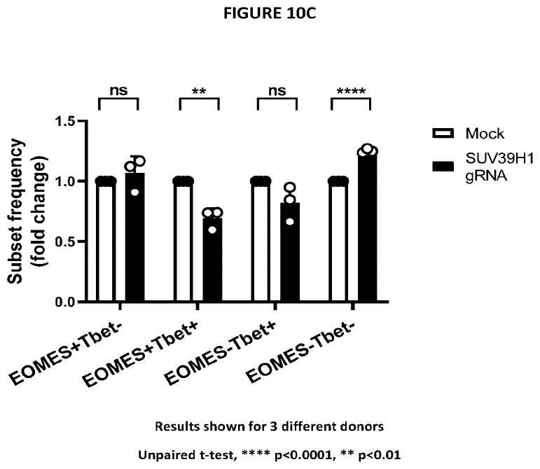

To observe the expression of central memory T cell surface markers important for the memory phenotype of CD8+ T cells, the SUV39H1KO CAR T cells from Example 7 were stimulated with aCD3+aCD28 beads for one week and then analyzed by flow cytometry. This allowed for the specific observation of the effect of SUV39H1 deletion on the proliferation of CAR T cells. Each week thereafter, the memory phenotype, gene expression profiles and cell numbers of SUV39H1 KO CAR T cells were analyzed. After one round of weekly stimulation, the fraction of CCR7+ CD45RO+ CD27+ CD62L+ cells, which constitute the Central Memory Cell subset, was increased in SUV39H1 KO CAR T cells. Results are shown in FIG. 9A as percentage of the cells that exhibit the Central Memory Cell phenotype compared to Mock per donor. FIG. 9B shows the fold change in the Central Memory Cells subset compared to Mock. Additionally, knocking out SUV39H1 in CAR T cells resulted in decreased expression levels of TIM-3 (FIG. 10A), decreased expression levels of T-bet (FIG. 10B), and increased frequency of T-bet-negative cells (FIG. 10C). Similar results were obtained for SUV39H1KO CAR T cells prepared using total CD3+ cells.

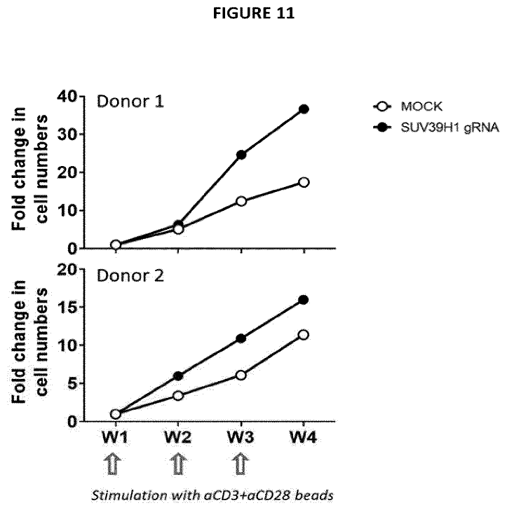

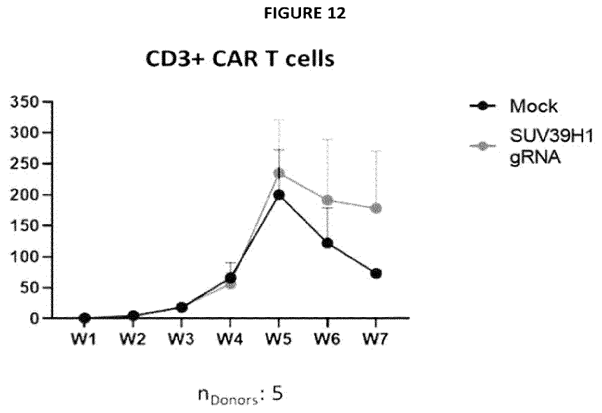

The cells were stimulated with aCD3+aCD28 beads once a week for a duration of 4 weeks. The fold change in cell numbers and kinetics during weekly stimulations of SUV39H1KO CD8+ CAR T cells are depicted in FIGS. 11 and 12 and show that these SUV39H1KO CAR T cells displayed increased in vitro proliferation and persistence after serial stimulations, a key predictor of in vivo anti-tumor efficacy.

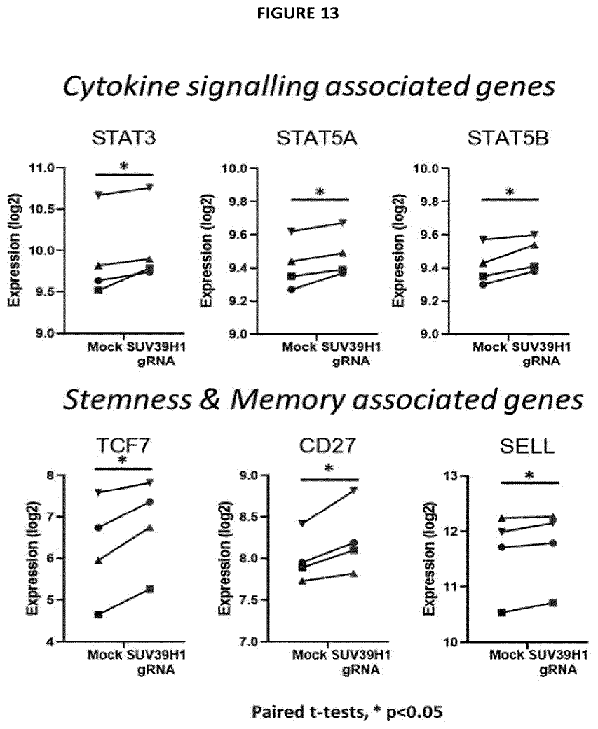

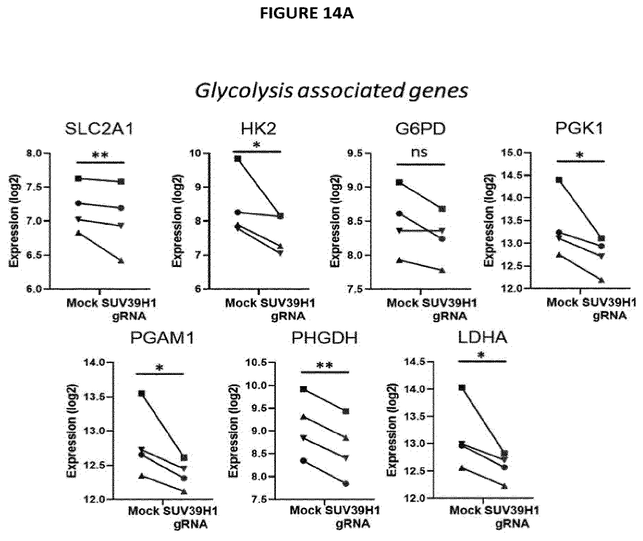

Immediately following production and before the first stimulation, Nanostring analysis (which quantifies mRNA levels) of the CAR T cell transcriptome was performed in four different donors. FIG. 13 illustrates that SUV39H1KO CAR T cells showed increased expression levels of the transcription factors STAT3, STAT5A and STAT5B. These transcription factors operate downstream of cytokine receptors, of the transcription factor TCF7, which promotes stemness and memory formation, and of the central memory markers CD27 and SELL (which encodes for CD62L). These results confirm that SUV39H1KO CAR T cells have a stem cell-like phenotype and are more receptive to cytokine signalling.

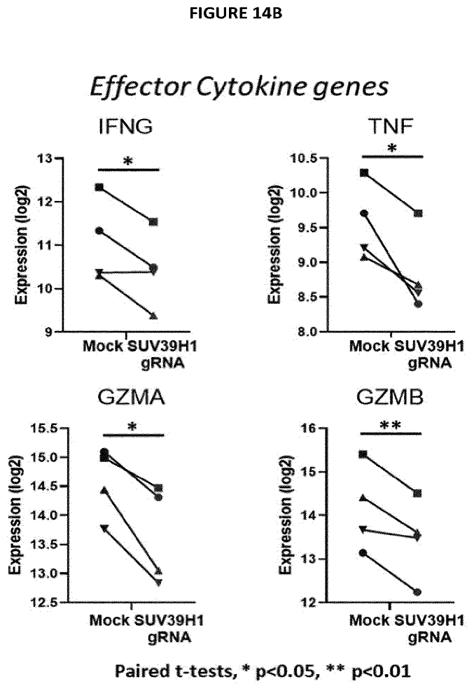

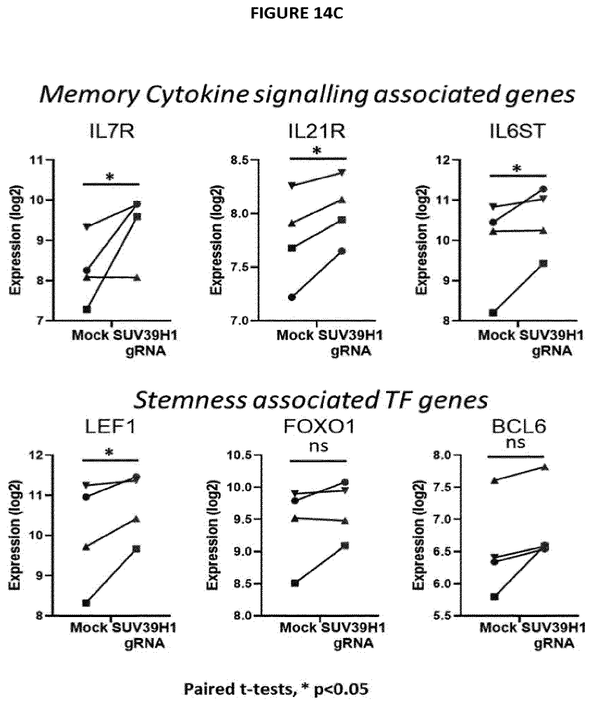

Nanostring analysis of CAR T cells after one round of weekly stimulation revealed that SUV39H1KO CAR T cells expressed lower levels of glycolytic enzymes (FIG. 14A) and effector cytokines (FIG. 14B). In contrast, FIG. 14C shows the CAR T cells had increased levels of cytokine receptor genes IL7R, IL21R and IL6ST, which are related to memory functions, and of stemness associated transcription factors, particularly LEF1. These results suggest that SUV39H1KO CAR T cells are less differentiated after one round of weekly stimulation compared to Mock.

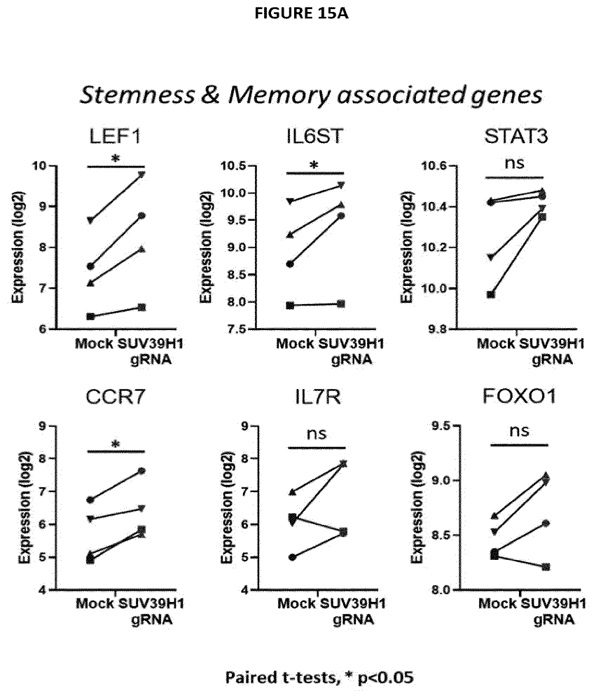

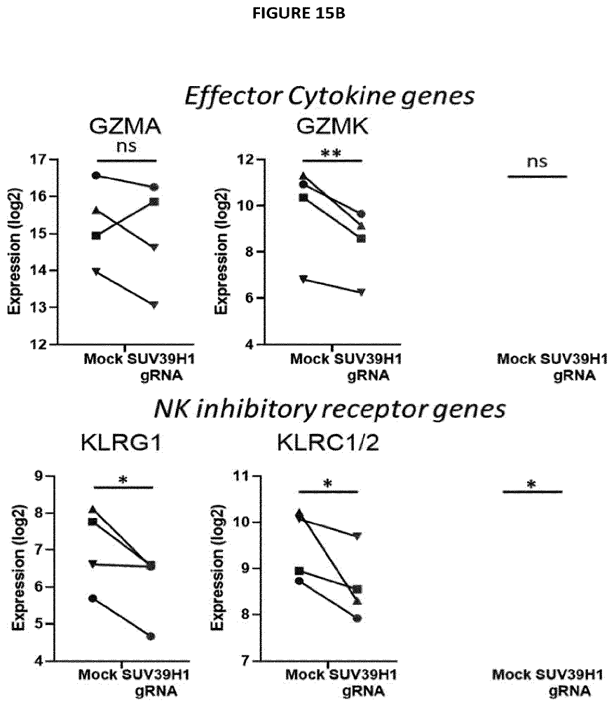

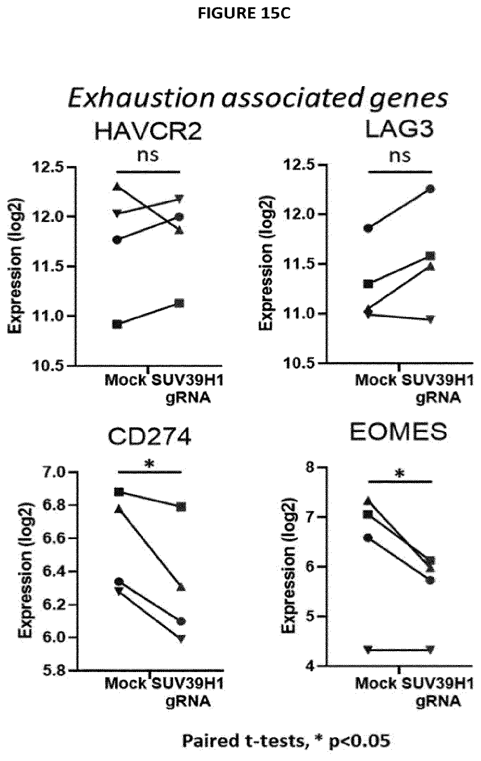

After three rounds of weekly stimulation, SUV39H1KO CAR T cells had increased expression levels of stemness and memory associated genes (FIG. 15A), and decreased expression levels of effector cytokines and inhibitory Natural Killer cell receptors (FIG. 15B), suggesting decreased terminal effector differentiation. Finally, SUV39H1KO and Mock CAR T cells do not have significant differences in the expression levels of the exhaustion markers HAVCR2 (encoding for TIM-3) and LAG3, but for CD274 (encoding for PD-L1) and EOMES (FIG. 15C). These results support a conclusion that SUV39H1KO CAR T cells are more resistant to terminal differentiation than Mock CAR T cells and maintain stem cell and memory characteristics.

### Example 9—Cytotoxic Function of SUV39H1KO CAR T Cells

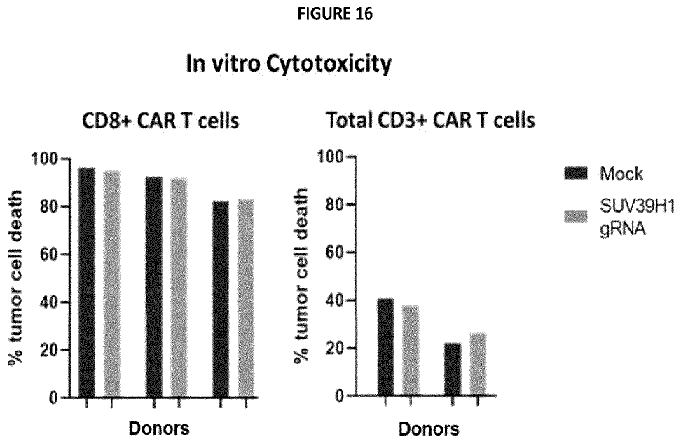

The effect of SUV39H1 deletion on the cytotoxicity of CAR T cells was evaluated by an in vitro killing assay against NALM-6 cells expressing luciferase. Briefly, 5×10⁴ NALM-6 cells were added in U-bottom plates and then effector cells were added at a 2:1 effector:target ratio. The plates were cultured overnight and, after the addition of luciferin, the bioluminescence of survived NALM-6 cells was quantified with a plate reader. No significant differences were found between the cytotoxic function of SUV39H1KO and Mock CAR T cells from either total CD3+ or purified CD8+ (FIG. 16).

### Example 10—Metabolic Fitness of SUV39H1KO CAR T Cells

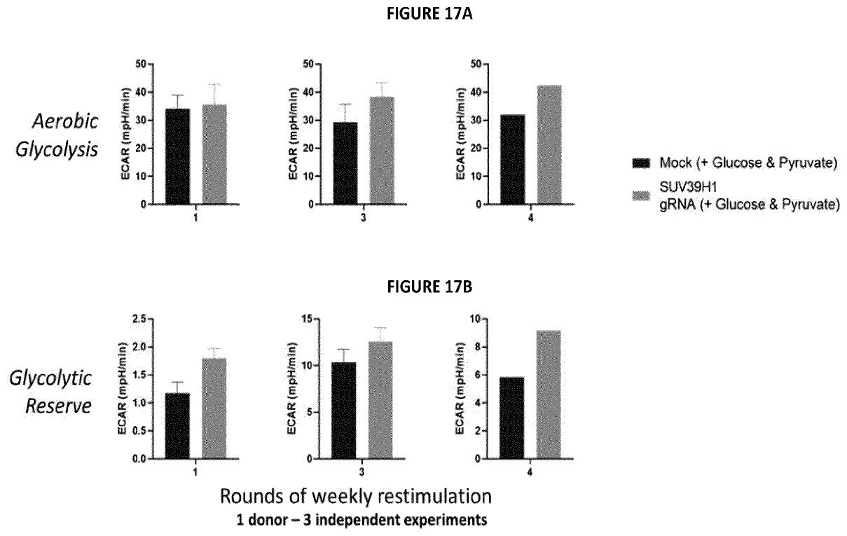

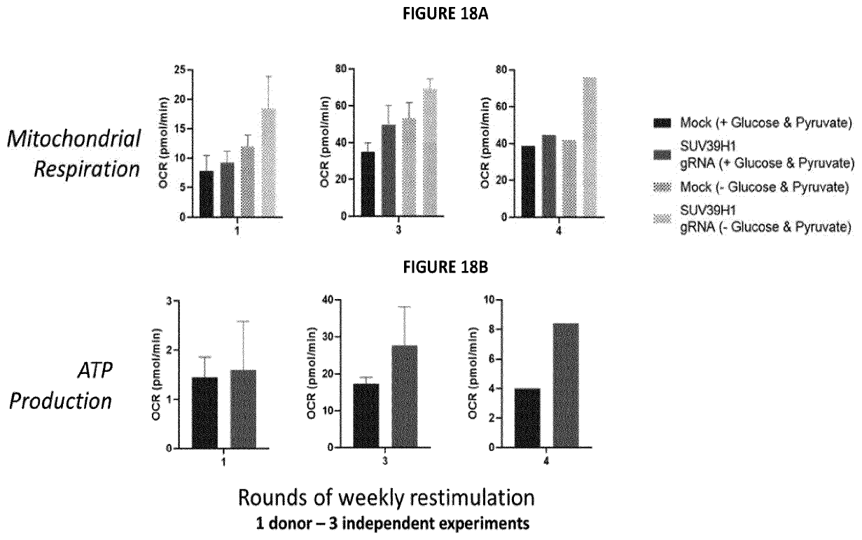

The metabolic characteristics of SUV39H1KO CAR T cells were examined at different time points during weekly stimulations using a commercial extracellular flux analyzer (Seahorse, Agilent). The extracellular acidification rate, a measure of aerobic glycolysis, and the oxygen consumption rate, a measure of mitochondrial respiration, were quantified. Briefly, 1.5×10⁵ cells were added per well and two different assays were performed in the analyzer. One assay was performed in the presence and the other assay in the absence of glucose and pyruvate, the initial substrates of glycolysis and mitochondrial respiration, respectively. It was determined that in the presence of glucose and pyruvate, SUV39H1KO CAR T cells were engaged in aerobic glycolysis at a similar extent to Mock (FIG. 17A) but showed marginally increased glycolytic reserve (calculated after the addition of the mitochondrial respiration inhibitor oligomycin and corresponding to this specific increase in extracellular acidification rate) (FIG. 17B). Similarly, in the presence of glucose and pyruvate, SUV39H1KO and Mock CAR T cells were also engaged in similarly efficient mitochondrial respiration (FIG. 18A). However, in the absence of glucose and pyruvate, SUV39H1KO CAR T cells had increased mitochondrial respiration (FIG. 18A). Finally, quantification of mitochondrial ATP production (calculated in the presence of glucose and pyruvate after the addition of the respiration inhibitor oligomycin and corresponding to this specific decrease in oxygen consumption rate) showed that, after more than three rounds of weekly stimulation, SUV39H1KO CAR T can continue to produce more ATP with this pathway (FIG. 18B). These results are consistent with the idea that SUV39H1KO CAR T cells are more metabolically fit than Mock CAR T cells and are more flexible in switching energy source in adverse conditions, namely switching to more glycolysis after inhibition of mitochondrial respiration or increasing mitochondrial respiration in the lack of glucose and pyruvate.

### Example 11—In Vivo Anti-Tumor Efficacy of Human SUV39H1 CAR T Cells

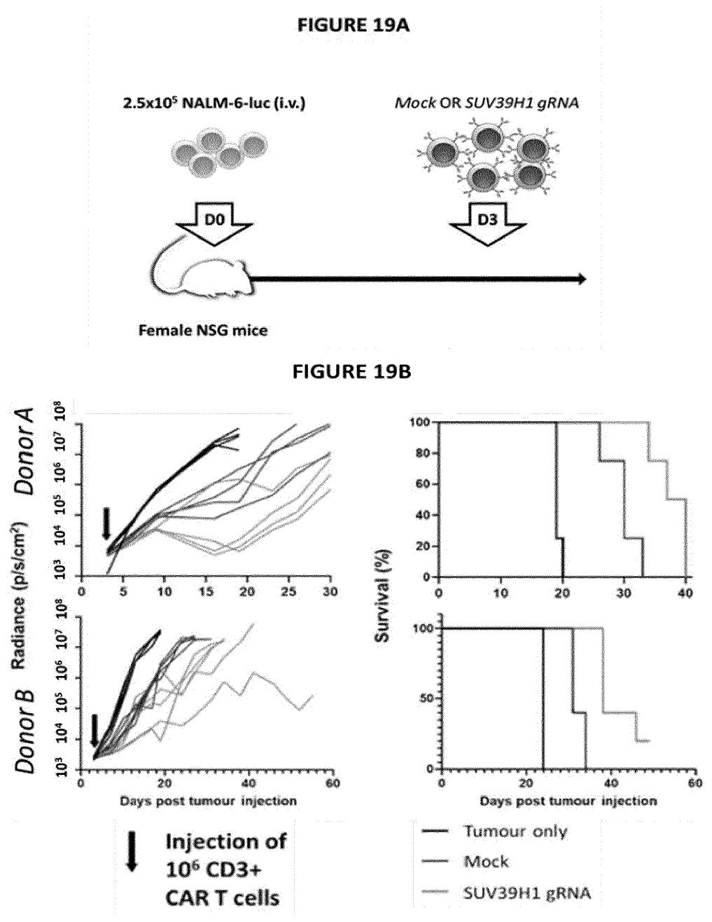

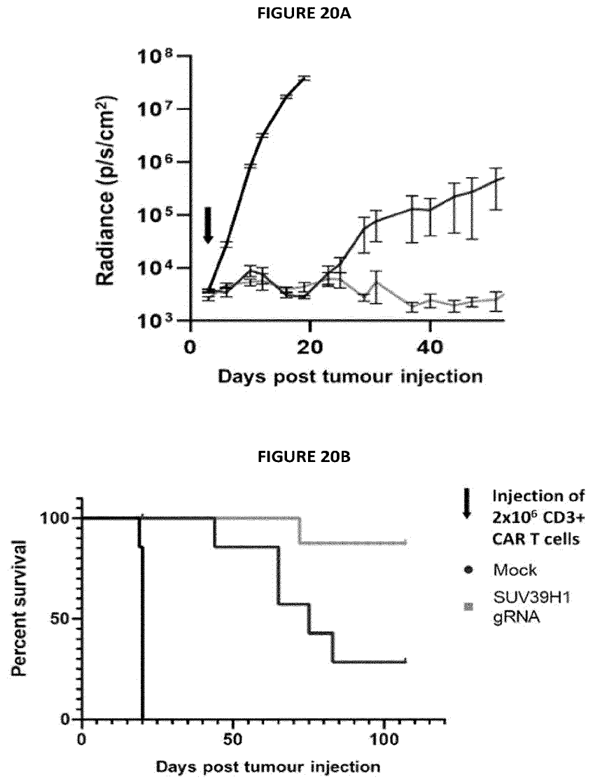

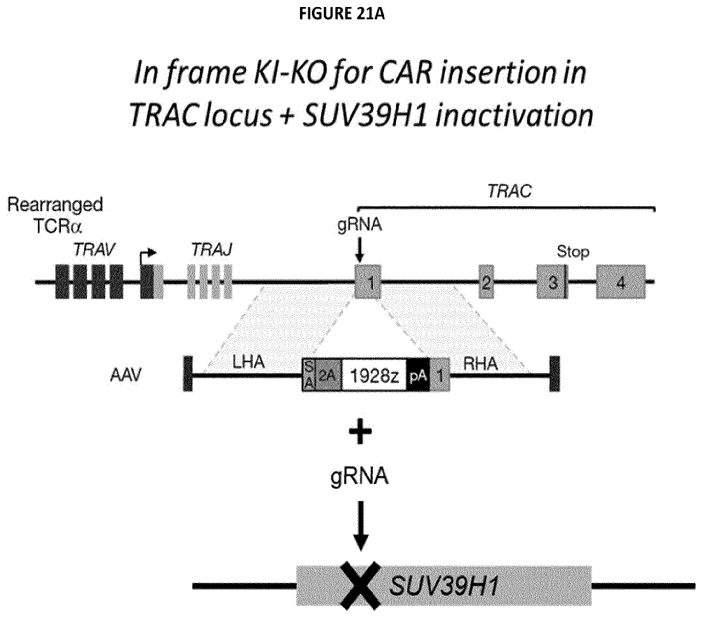

A xenogeneic model of acute lymphoblastic leukemia was used to study the effect of SUV39H1 on anti-tumor efficacy of human CAR T cells (FIG. 19A). Briefly, 2.5×105 NALM-6 cells expressing luciferase were injected intravenously in the tail of NSG mice and their growth in vivo was followed longitudinally by bioluminescence (IVIS, Perkin Elmer). On Day 3 post tumor injection, 10⁶ CAR T cells, either Mock or SUV39H1KO, were infused. For two different donors, SUV39H1KO CAR T cells displayed stronger anti-tumor response and enhanced the survival of NSG mice (FIG. 19B). Increasing the dose of CAR T cells to 2×10⁶ resulted in complete tumor rejection (FIG. 20A) and survival of 9 out of 10 mice (FIG. 20B).

### Example 12—Generation of CAR T Cells with Inactivated Endogenous TCR and Inactivated SUV39H1

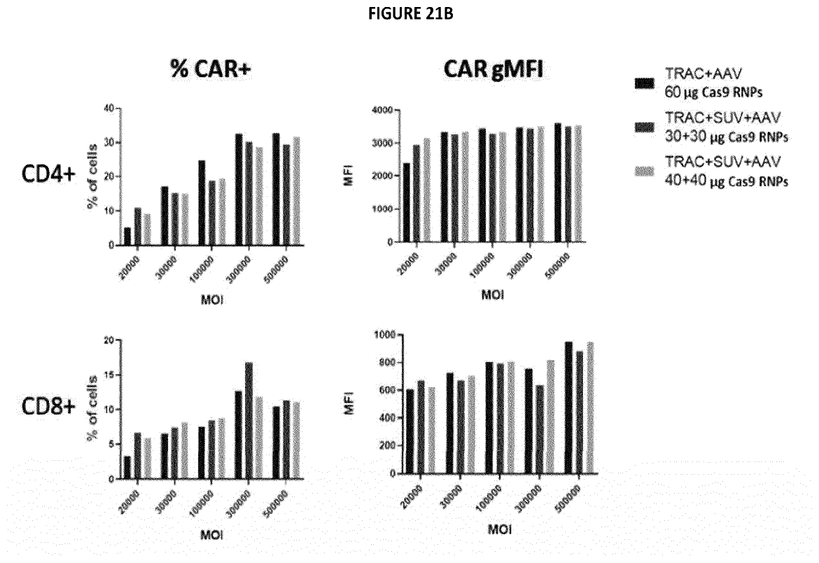

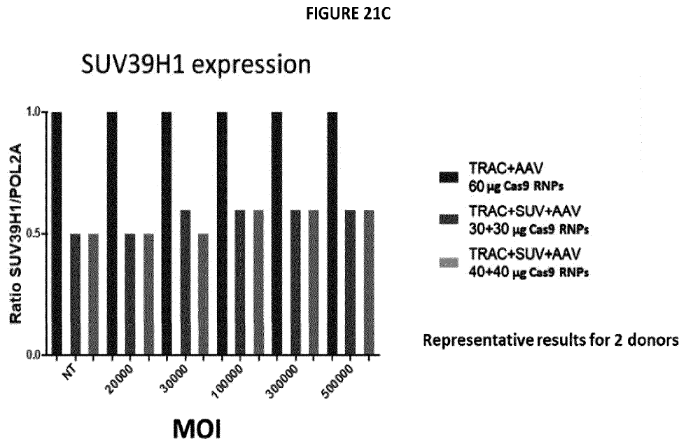

CRISPR-Cas9 RNPs were used to introduce the CAR gene into the T-cell receptor α constant (TRAC) locus, resulting in T cells that have significantly reduced or nearly eliminated expression of endogenous TCR, as shown in Eyquem et al., Nature 543: 113-117 (2017). The procedure is depicted in FIG. 21A.

Human T cells were electroporated with (1) Cas9 RNPs that contain gRNAs that target the first exon of the TRAC locus, preferably near the 5′ end (example of gRNA sequence:

5′C*A*G*GGUUCUGGAUAUCUGUGUUUUAGAGCUAGAAAUAGCAAGU
UAAAAUAAGGCUAGUCCGUUAUCAACUUGAAAAAGUGGCACCGAGUCG
GUGCU*U*U*U3′,

wherein the asterisk represents 2′-O-methyl 3′ phosphorothioate) (SEQ ID NO: 16), and (b) a donor AAV encoding an anti-CD19 CAR. The resulting T cells express the CAR under the control of the endogenous TRAC promoter. The T cells were also electroporated in parallel with Cas9 RNPs containing SUV39H1-targeting gRNAs that targeted exons of the SUV39H1 gene for deletion. Cells that express the anti-CD19 CAR were selected and expanded. FIGS. 21B-C show that such cells exhibit reduced expression of SUV39H1 and reduced expression of endogenous TCR.

FIG. 21B shows the percentage of CD4+ and CD8+ T cells that were CAR positive, at various titrated amounts of AAVs, and the geometric mean fluorescence intensity of CAR expression in the CAR+ cells. The latter showed stable CAR expression independently of the multiplicity of infection with AAVs, confirming the successful incorporation into the TRAC locus and control of CAR expression by the endogenous TRAC promoter. FIG. 21C shows the expression of SUV39H1 as measured by RT-qPCR. The results show successful and efficient deletion of SUV39H1 in the presence of the TRAC gRNA and independent of AAV transduction. Therefore, T cells that were produced with this protocol demonstrated both knock-in of the CAR in the TRAC locus and specific deletion of SUV39H1.

### Example 13—Generation of CAR T-Cells with Inactivated SUV39H1 and Reduced ITAM Activity

A nucleic acid is generated that encodes anti-CD19 CAR in which ITAM2 and ITAM3 are inactivated or deleted from the intracellular signaling region of CD3 zeta (ITAM-reduced CAR). The CAR has at least one co-stimulatory domain (e.g. CD28), or two or more co-stimulatory domains (CD27, CD28, 4-1BB, and/or OX40). The CAR-encoding nucleic acid is introduced into a human T-cell, and SUV39H1 is knocked out, according to Example 7 or Example 9.

Cells that express this anti-CD19 ITAM-reduced CAR and that exhibit reduced expression of SUV39H1 are thus generated using the methods of Example 7. Cells that express anti-CD19 ITAM-reduced CAR and that exhibit reduced expression of SUV39H1 and reduced expression of endogenous TCR are generated using the methods of Example 9. The resulting cells are evaluated for their Central Memory Cell phenotype (CCR7+CD45RO+CD27+CD62L+), proliferation after serial stimulation, and exhaustion characteristics (TIM-3, PD-1, LAG-3 expression).

1. A modified immune cell wherein the SUV39H1 gene is inactivated, said cell comprising:
a T cell receptor (TCR) alpha constant region gene inactivated by the insertion of a nucleic acid sequence encoding an antigen-specific receptor that specifically binds to an antigen, optionally a chimeric antigen receptor (CAR) or a heterologous TCR
wherein optionally the antigen is orphan tyrosine kinase receptor ROR1, tEGFR, Her2, p95HER2, LI-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, BCMA, Lewis Y, MAGE-A1, mesothelin, MUC1, MUC16, PSCA, NKG2D Ligands, NY-ESO-1, MART-1, gplOO, oncofetal antigen, TAG72, VEGF-R2, carcinoembryonic antigen (CEA), prostate specific antigen (PSMA), estrogen receptor, progesterone receptor, ephrinB2, CD 123, CS-1, c-Met, GD-2, MAGE A3, CE7, or Wilms Tumor 1 (WT-1).

2. A modified immune cell comprising a nucleic acid encoding a SUV39H1 inhibitor, optionally a dominant negative SUV39H1 gene, said cell comprising:
a T cell receptor (TCR) alpha constant region gene inactivated by the insertion of a nucleic acid sequence encoding an antigen-specific receptor that specifically binds to an antigen, optionally a chimeric antigen receptor (CAR) or a heterologous TCR
wherein optionally the antigen is orphan tyrosine kinase receptor ROR1, tEGFR, Her2, p95HER2, LI-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, BCMA, Lewis Y, MAGE-A1, mesothelin, MUC1, MUC16, PSCA, NKG2D Ligands, NY-ESO-1, MART-1, gplOO, oncofetal antigen, TAG72, VEGF-R2, carcinoembryonic antigen (CEA), prostate specific antigen (PSMA), estrogen receptor, progesterone receptor, ephrinB2, CD 123, CS-1, c-Met, GD-2, MAGE A3, CE7, or Wilms Tumor 1 (WT-1).

3. A modified immune cell according to claim 1, wherein the SUV39H1 gene is inactivated and that expresses a chimeric antigen receptor (CAR) comprising:
a) an extracellular antigen-binding domain that specifically binds an antigen,
b) a transmembrane domain,
c) optionally one or more costimulatory domains
d) an intracellular signaling domain comprising a modified CD3zeta intracellular signaling domain in which ITAM2 and ITAM3 have been inactivated,
wherein optionally the antigen is orphan tyrosine kinase receptor ROR1, tEGFR, Her2, p95HER2, LI-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, BCMA, Lewis Y, MAGE-A1, mesothelin, MUC1, MUC16, PSCA, NKG2D Ligands, NY-ESO-1, MART-1, gplOO, oncofetal antigen, TAG72, VEGF-R2, carcinoembryonic antigen (CEA), prostate specific antigen (PSMA), estrogen receptor, progesterone receptor, ephrinB2, CD 123, CS-1, c-Met, GD-2, MAGE A3, CE7, or Wilms Tumor 1 (WT-1).

4. A modified immune cell according to claim 2, comprising a nucleic acid encoding a SUV39H1 inhibitor, optionally a dominant negative SUV39H1 gene, and that expresses a chimeric antigen receptor (CAR) comprising:
a) an extracellular antigen-binding domain that specifically binds an antigen,
b) a transmembrane domain,
c) optionally one or more costimulatory domains
d) an intracellular signaling domain comprising a modified CD3zeta intracellular signaling domain in which ITAM2 and ITAM3 have been inactivated,
wherein optionally the antigen is orphan tyrosine kinase receptor ROR1, tEGFR, Her2, p95HER2, LI-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, BCMA, Lewis Y, MAGE-A1, mesothelin, MUC1, MUC16, PSCA, NKG2D Ligands, NY-ESO-1, MART-1, gplOO, oncofetal antigen, TAG72, VEGF-R2, carcinoembryonic antigen (CEA), prostate specific antigen (PSMA), estrogen receptor, progesterone receptor, ephrinB2, CD 123, CS-1, c-Met, GD-2, MAGE A3, CE7, or Wilms Tumor 1 (WT-1).

5. The modified immune cell of claim 1, wherein the cell is a T cell, a T cell progenitor, a hematopoietic stem cell, an iPSC, a CD4+ T cell, a CD8+ T cell, a CD4+ and CD8+ T cell, or a NK cell, or a T_{N} cell, T_{SCM}, T_{CM} or T_{EM} cell or a T regulatory cell.

6. (canceled)

7. The modified immune cell of claim 1, wherein the CAR comprises: (a) an extracellular antigen-binding domain; (b) a transmembrane domain, (c) optionally one or more costimulatory domains, and (d) an intracellular signaling domain.

8. The modified immune cell of claim 7, wherein:
(a) the extracellular antigen-binding domain is optionally an scFv, optionally an scFv that specifically binds a cancer antigen;
(b) the transmembrane domain is optionally selected from the group consisting of: CD28, CD8 and CD3-zeta;
(c) the one or more costimulatory domains are optionally selected from the group consisting of: 4-1BB, CD28, ICOS, OX40 and DAP10; and
(d) the intracellular signaling domain optionally comprises the intracellular signaling domain of a CD3-zeta polypeptide, or a fragment thereof, optionally a CD3-zeta polypeptide wherein immunoreceptor tyrosine-based activation motif 2 (ITAM2) and immunoreceptor tyrosine-based activation motif 3 (ITAM3) are inactivated.

9-11. (canceled)

12. The modified immune cell of claim 1, wherein the T cell further comprises a second antigen-specific receptor, optionally a TCR or CAR, that specifically binds to a second antigen.

13. The modified immune cell of claim 1, wherein SUV39H1 expression is reduced by at least about 50%, 60%, 70%, 75%, 80%, 85%, 90% or 95%.

14. The modified immune cell of claim 1, wherein endogenous TCR expression is reduced by at least about 75%, 80%, 85%, 90% or 95%.

15. The modified immune cell of claim 1, wherein the immune cell is autologous or allogeneic.

16. (canceled)

17. The modified immune cell of claim 1, wherein the HLA-A locus is inactivated.

18. The modified immune cell of claim 17 wherein HLA class I expression is reduced by at least about 75%, 80%, 85%, 90% or 95%.

19. The modified immune cell of claim 1 that expresses two CARs, a first CAR that binds a first antigen and a second CAR that binds a second antigen.

20. A sterile pharmaceutical composition comprising the modified immune cell of claim 1.

21. A kit comprising the modified immune cell of claim 1, and a delivery device or container.

22. A method of using the modified immune cell of claim 1 to treat a patient suffering from or at risk of disease associated with the antigen, optionally cancer, by administering a therapeutically effective amount of said immune cell or pharmaceutical composition to the patient.

23. The method of claim 22 wherein the immune cell is a CAR T-cell and a dose of less than about 5×10⁷ cells, optionally about 10⁵ to about 10⁷ cells, is administered to the patient.

24. The method of claim 22 wherein a second therapeutic agent, optionally one or more cancer chemotherapeutic agents, cytotoxic agents, hormones, anti-angiogens, radiolabelled compounds, immunotherapy, surgery, cryotherapy, and/or radiotherapy, or optionally an immune checkpoint modulator is administered to the patient.

25. (canceled)

26. The method of claim 24, wherein the immune checkpoint modulator is an antibody that specifically binds to, or other inhibitor of, PD1, PDL1, CTLA4, LAG3, BTLA, OX2R, TIM-3, TIGIT, LAIR-1, PGE2 receptor, EP2/4 adenosine receptor, or A2AR.

27. The modified immune cell of claim 2, wherein the cell is a T cell, a T cell progenitor, a hematopoietic stem cell, an iPSC, a CD4+ T cell, a CD8+ T cell, a CD4+ and CD8+ T cell, or a NK cell, or a T_{N} cell, T_{SCM}, T_{CM} or T_{EM} cell or a T regulatory cell.
